SEQUENCE LISTING (28 sequences)

SEQ ID NO: 1 - protein, 11 aa, Homo sapiens
RERTELLLADY

SEQ ID NO: 2 - protein, 23 aa, Homo sapiens
MADPLRERTELLLADYLGYCARE

SEQ ID NO: 3 - protein, 22 aa, Homo sapiens
ADPLRERTELLLADYLGYCARE

SEQ ID NO: 4 - protein, 36 aa, Artificial Sequence
  Synthetic peptide TAT-ESP-peptide
RKKRRQRRRGGSGGADPLRERTELLLADYLGYCARE

SEQ ID NO: 5 - protein, 23 aa, Artificial Sequence
  Synthetic peptide Peptide R6A
MADPLAERTELLLADYLGYCARE

SEQ ID NO: 6 - protein, 23 aa, Artificial Sequence
  Synthetic peptide Y16F
MADPLRERTELLLADFLGYCARE

SEQ ID NO: 7 - protein, 23 aa, Artificial Sequence
  Synthetic peptide D15A
MADPLRERTELLLAAYLGYCARE

SEQ ID NO: 8 - protein, 23 aa, Artificial Sequence
  Synthetic peptide C20A
MADPLRERTELLLADYLGYAARE

SEQ ID NO: 9 - protein, 11 aa, Artificial Sequence
  Synthetic peptide
  misc_feature (8)..(9): Xaa can be any naturally occurring amino acid
RERTELLXXDY

SEQ ID NO: 10 - protein, 11 aa, Artificial Sequence
  Synthetic peptide
  misc_feature (5)..(6): Xaa can be any naturally occurring amino acid
RERTXXLLADY

SEQ ID NO: 11 - protein, 11 aa, Artificial Sequence
  Synthetic peptide
  misc_feature (3)..(3): Xaa can be any naturally occurring amino acid
  misc_feature (10)..(10): Xaa can be any naturally occurring amino acid
REXTELLLAXY

SEQ ID NO: 12 - protein, 9 aa, Human immunodeficiency virus
RKKRRQRRR

SEQ ID NO: 13 - protein, 16 aa, Drosophila
RQIKIWFQNRRMKWKK

SEQ ID NO: 14 - protein, 5 aa, Artificial Sequence
  Spacer
GGSGG

SEQ ID NO: 15 - protein, 26 aa, Homo sapiens
WWTNWVIPAISAVAVALMYRLYMAED

SEQ ID NO: 16 - protein, 4 aa, Artificial Sequence
  ER addressing
KDEL

SEQ ID NO: 17 - protein, 4 aa, Artificial Sequence
  ER addressing
KKMP

SEQ ID NO: 18 - protein, 62 aa, Artificial Sequence
  Synthetic peptide TAT-ESP-peptide-Cb5
RKKRRQRRRGGSGGADPLRERTELLLADYLGYCAREWWTNWVIPAISAVAVALMYRLYMAED

SEQ ID NO: 19 - protein, 27 aa, Danio rerio
MSCWLREQTLLLAEDYISFCSGIQQTP

SEQ ID NO: 20 - protein, 27 aa, Gallus gallus
MPGSLKEETALLLEDYFQHRAGGAALP

SEQ ID NO: 21 - protein, 30 aa, Mus musculus
MADSQDPLHERTRRLLSDYIFFCAREPDTP

SEQ ID NO: 22 - protein, 27 aa, Homo sapiens
MADPLRERTELLLADYLGYCAREPGTP

SEQ ID NO: 23 - protein, 29 aa, Homo sapiens
MAHAGRTGYDNREIVMKYIHYKLSQRGYE

SEQ ID NO: 24 - protein, 23 aa, Homo sapiens
MSQSNRELVVDFLSYKLSQKGYS

SEQ ID NO: 25 - protein, 27 aa, Homo sapiens
MTDCEFGYIYRLAQDYLQCVLQIPQPG

SEQ ID NO: 26 - protein, 27 aa, Homo sapiens
MATPASAPDTRALVADFVGYKLRQKGY

SEQ ID NO: 27 - protein, 38 aa, Artificial Sequence
  Synthetic peptide CK-TAT-ESP-peptide C20A
CKRKKRRQRRRGGSGGADPLRERTELLLADYLGYAARE

SEQ ID NO: 28 - protein, 38 aa, Artificial Sequence
  Synthetic peptide CK-TAT-ESP-peptide C20A Y16F
CKRKKRRQRRRGGSGGADPLRERTELLLADFLGYAARE

(19) United States
(12) Patent Application Publication
(10) Pub. No.: US 2019/0030126 A1
(43) Pub. Date: Jan. 31, 2019

(54) Inhibitors of the Interaction BCL-2 L10 / IP3 Receptors

(71) Applicants: Universite Claude Bernard - Lyon 1, Villeurbanne (FR); Centre Leon Berard, Lyon (FR); Centre National de la Recherche Scientifique (CNRS), Paris (FR); Insititut National de la Sante et de la Recherche Medicale (INSERM), Paris (FR)

(72) Inventors: Adrien Nougarede, Charly (FR); Ruth Rimokh, Lyon (FR); Germain Gillet, Lyon (FR); Nikolay Georgiev, Lyon (FR)

(73) Assignees: Universite Claude Bernard - Lyon 1, Villeurbanne (FR); Centre Leon Berard, Lyon (FR); Centre National de la Recherche Scientifique (CNRS ), Paris (FR); Institut National de la Sante et de la Recherche M edicale (INSERM), Paris (FR)

(21) Appl. No.: 16/081,258

(22) PCT Filed: Mar. 8, 2017
(86) PCT No.: PCT/EP2017/055469

§ 371 Date: Aug. 30, 2018

(30) Foreign Application Priority Data
Mar. 11, 2016 (FR) 1652040

(51) Int. Cl. A61K 38/17; A61P 35/00; A61K 38/04

(52) U.S. Cl. CPC A61K 38/1761; A61K 38/04; A61K 38/177; A61P 35/00

(57) ABSTRACT

The present invention relates to a competitive inhibitor of the binding of the protein Bcl-2 L10 to the ligand binding domain of at least one of the IP3R receptors, for its use in the treatment of cancers, the cells of which express the protein Bcl-2 L10.

### FIELD OF THE INVENTION

The present invention relates to the pharmaceutical field, and more particularly to the field of oncology, especially to the field of the treatment of cancers, the cells of which express the protein Bcl-2 L10.

### PRIOR ART

Cancer is a disease of humans and animals which is commonly fatal, and which is reflected by uncontrolled growth of endogenous cells. The term “cancer” is used to denote the formation of malignant tumours and neoplasms (tumours or carcinomas). Generally, the growth of cancers comprises two stages, namely local tumour growth then the spread of the cancer. Local tumour growth consists of the appearance of a tumour clone from a “transformed” cell, under the action of initiating and promoting carcinogenic agents. Cell proliferation leads to the formation of the tumour. The spread of the cancer from the initial tumour may occur by regional spread and/or by development of secondary tumours referred to as metastases.

In order to treat cancer, various techniques such as surgery, radiotherapy, chemotherapy, hormone therapy, immunotherapy and antiangiogenic therapy have been developed.

The choice of the most suited therapy or therapies is based on various factors and especially depends on the therapeutic protocol commonly followed for each type of cancer, on the stage of the illness at diagnosis, and on the general condition of the patient.

It appears however that the use of certain chemotherapy products intended to kill cancer cells by inducing their apoptosis may prove to be effective in some patients but ineffective in others.

Certain biomarkers have been identified, the presence or overexpression of which has been observed in patients presenting resistance to medicaments which are nonetheless known for their effectiveness in the pathological condition in question.

Thus, practitioners endeavour on the one hand to determine those patients liable to respond to certain treatments and those liable to be resistant to these treatments, and on the other hand to develop strategies making it possible to obtain reinforced effectiveness of the anti-cancer products in those patients having an insufficient response to conventional treatments. The present invention aims to satisfy this need for potentiation of existing treatments, especially in patients presenting resistance to these treatments.

Molecules of the Bcl-2 Family

“Chemotherapy” treatments are based on the administration of chemical molecules intended to induce apoptosis of cancer cells present in a patient.

Apoptosis, also referred to as “programmed cell death”, plays a pivotal role in numerous biological processes, both normal and pathological. Two predominant pathways of apoptosis are distinguished, the intrinsic, mitochondrial, pathway and the extrinsic pathway activated by specific extracellular signals.

The proteins of the Bcl-2 family are the main regulators of mitochondrial apoptosis. The membership of a protein to the Bcl-2 family is linked to the presence of one or more domains of peptide sequence homology with the protein initially identified, named BH domain (Bcl-2 Homology domains).

The proteins of the Bcl-2 family may regulate apoptosis by promoting (pro-apoptotic) or by inhibiting (anti-apoptotic) programmed cell death. This family of proteins is divided into three subgroups:
pro-apoptotic effector proteins such as Bax and Bak, which, once activated by cell signals, insert into the outer mitochondrial membrane to cause the release of cytochrome C, thereby inducing apoptosis.
anti-apoptotic proteins such as Bcl-2, Bcl-xL, Mcl-1, Bcl-2A1, Bcl-W, and Bcl-B/Bcl-2 L10, which inhibit apoptosis by interacting directly with the pro-apoptotic proteins to neutralize them. The hydrophobic interaction pocket formed by the domains BH3, BH1 and BH2 of the anti-apoptotic proteins enables binding of the BH3 domain to the Bax and Bak proteins, thereby leading to their neutralization. These proteins also have a fourth domain BH4 involved in the regulation of calcium fluxes.
BH3-only proteins, which only contain a single homology domain BH3. These BH3-only proteins bind in the hydrophobic pocket of the anti-apoptotic proteins, preventing them from neutralizing Bax and Bak. Some therapies use a similar chemical structure to the BH3 of the BH3-onlys to inhibit the anti-apoptotic Bcl-2 proteins and to induce cell death. These molecules are referred to as BH3 mimetics (Besbes et al., 2015).

The proteins of the Bcl-2 family regulate apoptosis, especially by promoting or inhibiting the permeability of the outer mitochondrial membrane to cytochrome C. The release of cytochrome C into the cytosol then leads to the activation of caspases (cysteine-aspartic proteases), proteases which are responsible for the enzymatic cleavage of numerous cellular proteins thereby inducing cell death.

Other stimuli may also trigger the release of cytochrome C, such as a massive influx of calcium from the endoplasmic reticulum to the cytosol then the mitochondria.

Over the course of tumorigenesis, overexpression of the anti-apoptotic proteins is commonly observed, which enables the tumour cells to evade the organism's endogenous controls and also the medicaments intended to induce their cell death.

The importance of the biological processes controlled by the Bcl-2 family makes them a highly studied protein family. However, in the group of the anti-apoptotics, the function of some of them remains poorly understood; this is especially the case for the product of the gene bcl2-l10/bcl-b/nrh.

Bcl-2 L10

The gene bcl-2 l10 is evolutionarily conserved, in particular in vertebrates (Arnaud et al, 2006).

In humans, the expression of bcl-2 l10 in normal adult tissues is restricted to the oocytes, the ovaries and the B-lymphocyte cells. In mice, its knockout did not give rise to an observable phenotype.

In the zebrafish, its expression is observed in the early stages of development. In this animal model, in which the gene bcl-2 l10 is referred to as nrz, this gene is indispensable to embryonic development, its knockout causing a lethal phenotype. It has been shown that the protein Nrz is able to regulate calcium trafficking in the cell by interacting, at the endoplasmic reticulum, with IP3R1, inositol 1,4,5-triphosphate (IP3)-sensitive calcium channel (Popgeorgiev et al., 2011).

The protein Bcl-2 L10/Nrz is able to interact with the domain for binding of the ligand IP3 of the receptor IP3R1 (IP3BD) via its BH4 domain. The recombinant protein Nrz has proven capable of inhibiting the binding of IP3 to the receptor IP3R1. The BH4 domain is required for the interaction with the receptor IP3R1, but is not sufficient alone to inhibit the release of Ca²⁺ via IP3R1 (Bonneau et al., 2014).

In a disease context, in humans, expression of the gene bcl-2 l10 is correlated with a poor prognosis in breast, prostate and lung tumours, acute myeloid leukaemias and myelodysplastic syndromes (Krajewska et al., 2008; Cluzeau et al., 2012). The gene bcl-2 l10 is thus considered a marker of a poor prognosis for these tumour-based pathological conditions. Detection of the protein produced by this gene as a diagnostic test of azacitidine resistance in haematopoietic tumours was the subject of a patent application (WO2013/128089 A1).

### IP3R Receptors

Different proteins of the Bcl-2 family are able to prevent the process of programmed cell death, especially by negatively regulating calcium fluxes.

Calcium (Ca²⁺) is a universal intracellular messenger which regulates highly diverse cellular activities such as cell death or differentiation through a highly developed signalling network, from extracellular and intracellular sources of Ca²⁺.

Inositol 1,4,5-triphosphate receptors, referred to hereinafter as “IP3R”, are tetrameric calcium channels located on the membrane of the endoplasmic reticulum, present in all cells. These channels control the release of calcium from this compartment to the cytosol, in response to numerous signals induced especially by tyrosine kinase receptors and G-protein coupled plasma membrane receptors, responsible for variations in the concentration of IP3, the ligand which activates IP3Rs. High concentrations of IP3 lead to a significant release of calcium ions into the cytosol.

Three types of IP3R receptor have been identified, and are denoted IP3R1, IP3R2 and IP3R3. These large receptors, comprising more than 2500 amino acids, are divided into several domains (see FIG. 1).

The protein Bcl-2 interacts via its BH4 domain with the receptor IP3R1 at the MTD II domain (Rong et al., 2008) and in the C-terminal portion (Monaco et al., 2012). Just the presence of the BH4 domain is sufficient to block the release of calcium by the IP3R receptors (Rong et al., 2009). This has especially been demonstrated by the use of a peptide of 20 amino acids, mimicking the binding domain of the central portion of IP3R1 (amino acids 1389-1408), which interacts with the BH4 domain of Bcl-2 and thereby blocks its biological activities.

Using this peptide, it has been shown that it is possible to inhibit the function of the Bcl-2 protein in the regulation of calcium fluxes, and thereby to promote and/or induce apoptosis of different types of tumour cells (Zhong et al., 2011; Lavik et al., 2015). The use of this peptide as medicament, especially for treating cancers characterized by cellular expression of Bcl-2, has been proposed (WO2012/031103).

This strategy has the drawback of not being very specific, since numerous proteins of the Bcl-2 family are able to interact with this peptide mimicking the central domain of IP3R1, and therefore to be neutralized. In particular, it has been shown that this binding site is also used by the protein Bcl-X_{L} (Monaco et al., 2012(1)). However, the action of this protein involves different biological mechanisms from those mediated by Bcl-2 (Monaco et al., 2012(2)).

Moreover, the use of such a peptide derived from IP3R1, potentially capable of non-specifically neutralizing other signalling pathways, could induce significant side effects.

Despite its involvement in tumorigenesis, and its role as a diagnostic tool, the function of the protein encoded by bcl2-l10 remains poorly understood and as yet no solution has been found for specifically inhibiting and targeting the activity of this protein which promotes the survival of tumour cells, including when patients are subjected to treatments based on chemotherapy compounds.

Compounds making it possible to block the biological function of the protein Bcl-2 L10 when the latter binds to the IP3R receptor, and thereby blocks the influx of calcium to the cytosol which should be triggered by a chemotherapy treatment, are being actively sought in order to potentiate the effects of chemotherapy treatment.

These compounds must advantageously have several characteristics: prevent the binding of Bcl-2 L10 to the IP3R receptor, but without inhibiting the binding of the ligand IP3 to its receptor; not inhibit the other biological functions of Bcl-2 L10; and not inhibit the other proteins of the Bcl-2 family.

The present application presents compounds which meet these requirements.

### SUMMARY OF THE INVENTION

The present invention relates to a competitive inhibitor of the binding of the protein Bcl-2 L10 to the IP3 ligand binding domain of at least one of the IP3R receptors, for its use in the treatment of cancers, the cells of which express the protein Bcl-2 L10.

The present invention most particularly relates to an inhibitor, for its use in the treatment of said cancers, which comprises a peptide domain, the sequence of which has at least 80% identity with the sequence SEQ ID NO. 1 [RERTELLLADY], the underlined arginine and tyrosine residues being conserved.

According to a particular aspect of the invention, this inhibitor is administered in combination with at least a second active agent and/or any conventional method for treating cancer, in particular is administered in combination with at least one chemotherapy product for the treatment of cancers, the cells of which express the protein Bcl-2 L10.

The present invention also relates to a competitive inhibitor of the binding of the protein Bcl-2 L10 to the IP3 ligand binding domain of at least one of the IP3R receptors constructed according to the following structure: [ADR]ₓ-[ESP]_{y}-[DOM]-[SADR]_{z}, in which
ADR is a targeting compound,
ESP is a spacer,
DOM is the peptide domain defined in Claim 2 or 3,
SADR denotes a specific intracellular targeting peptide domain,

wherein x, y and z are equal to 0 or 1 independently of one another, and the sum (x+y+z) is equal or superior to 1.

The present invention also relates to a pharmaceutical composition comprising, in a pharmaceutically acceptable medium, at least one competitive inhibitor of the binding of the protein Bcl-2 L10 to the IP3 ligand binding domain of at least one of the IP3R receptors, as described above.

### LEGEND OF THE FIGURES

FIG. 1: Diagram of the primary structure of the human IP3 receptor type 1 (hIP3R1), split into seven functional domains: SD (Suppressor Domain), IP3BD (IP3 ligand Binding Domain), MTD (Modulatory and Transducing Domain), CFD (Channel Forming Domain) and CD (Coupling Domain). The proteins Bcl-2 and Bcl-xL interact with the domains MTD II and Coupling Domain. The protein Nrh interacts with the IP3 binding domain (IP3BD). Peptides binding to this receptor have been described previously, but have different binding sites, as indicated.

FIG. 2: Immunoblot of the endogenous protein Nrh from total, mitochondrial (Mito) or reticular (ER) fractions or fractions that are soluble after centrifugation at 100000 G (S100), obtained from mammary carcinoma cells MDA-MB-231. The proteins vinculin, calnexin and FOF1 are used as control.

FIG. 3: Kinetics of apoptosis (activity of caspases 3 and 7) in response to 10 μM of thapsigargin (THG) over 36 h, of MDA-MB-231 cells transfected by siRNAs against Nrh (si3-Nrh and si6-Nrh), or with a control siRNA (siSCR). Incubation with DMSO serves as negative control for incubation with thapsigargin.

FIG. 4:

(A) Diagram of the constructs produced from the gene nrh and targeting different cellular compartments: the mitochondria (Nrh-ActA, with mitochondrial targeting domain derived from the ActA protein of L. monocytogene), the endoplasmic reticulum (Nrh-cb5, with reticular targeting domain derived from the human Cytochrome b5 protein), the membrane (Nrh-dTM with its C-terminal membrane targeting domain), or Nrh without its N-terminal BH4 domain (Nrh-dBH4).

(B) Test of apoptosis (SR-FLICA) in response to 10 μM of thapsigargin for 24 h in HeLa cells transfected with the empty vector pCS2+ or the constructs coding for Nrh-WT (wild-type), Nrh-ActA, Nrh-cb5, Nrh-dTM or Nrh-dBH4 (N=3). ***, p<0.001;**, p<0.01; n.s. p>0.1 (Student t-test).

FIG. 5:

(A) Diagram of the peptide Nrh 1-23 and point mutants constructed from the BH4 domain of Nrh, fused with the targeting sequence Cb5.

(B) Quantification of the Nrh-IP3R1 interaction by immunoprecipitation experiments with anti-HA antibodies in HeLa cells transfected with plasmids coding for Flag-Nrh, HA-hBD and the different point mutants of the peptides Nrh 1-23 Flag-tagged with a ratio of 1:2 (N=3).

(C) Test of apoptosis (SR-FLICA) in response to 10 μM of thapsigargin for 24 h in HeLa cells transfected with the empty vector pCS2+ or the constructs coding for the peptides Nrh 1-23, Nrh 1-23 Y16F or Nrh 1-23 C20A (N=3). ***, p<0.001;**, p<0.01; n.s. p>0.1 (Student t-test).

FIG. 6:

(A) Streptavidin pull-down in HeLa cells transfected with either empty vector pCS2+, Flag-Nrh, HA-hBD or HA-BECN1, which cellular extracts were incubated or not with 5 μg of biotin-labelled Nrh 1-23 peptide. Nrh 1-23 is able to interact with Nrh, but not with HA-hBD nor HA-BECN1.

(B) Streptavidin pull-down in HeLa cells transfected with either empty vector pCS2+, Flag-Nrh, or Flag-Nrz, which cellular extracts were incubated with 5 μg of biotin-labelled Nrh 1-23 peptide or Nrh 1-23 R6A Y16F double mutant peptide. Nrh 1-23 is able to interact with Nrh, but not with Nrz, thus exhibiting species-specificity.

FIG. 7:

Quantification of the interaction between Flag-Nrh and HA-Nrh using co-immunoprecipitation with anti-HA antibody. Results show a Nrh homodimer formation. Nrh dimer can be prevented in presence of Nrh 1-23 peptide of the present invention, but not of Nrh 1-23 Y16F.

FIG. 8: Alignment of the amino acid sequences originating from the BH4 domains of different Nrh protein orthologs: BH4 domain from Nrz (Danio rerio, SEQ ID NO. 19), from NR-13 (Gallus gallus, SEQ ID NO. 20), from mouse Bcl-2 L10 (Mus musculus, SEQ ID NO. 21), from human Bcl-2 L10 (Nrh, SEQ ID NO. 22), from human Bcl-2 (SEQ ID NO. 23), from human Bcl-xL (SEQ ID NO. 24), from human Bcl-A1 (SEQ ID NO. 25), and from human Bcl-W (SEQ ID NO. 26). Critical amino acids for the Nrh/IP3R1 interaction are indicated with arrows.

FIG. 9:

(A) Sequences of the synthetic peptides Nrh 1-23 and Nrh 1-23 Y16F fused to a TAT internalization domain by means of a spacer (linker) and coupled to a fluorochrome (FITC) at the N-terminal.

(B) Circular dichroism spectra of the peptides TAT-Nrh 1-23 and TAT-Nrh 1-23 Y16F described in FIG. 9A, dissolved in [H₂O-0.1% trifluoroacetic acid] at a concentration of 80 μM in 20% trifluoroethanol, showing a characteristic signature of an alpha helix structure for each of the peptides.

FIG. 10:

(A) Proximity ligation assay (PLA) experiments allowing for the detection of the interaction between the endogenous proteins Nrh and IP3R1 in MDA-MB-231 cells.

(B) Test of apoptosis (SR-FLICA) in response to 10 μM of thapsigargin for 36 h in MDA-MB-231 cells incubated with the peptide TAT-Nrh 1-23, or TAT-Nrh 1-23 Y16F, after a flash of light (λ 488 nm) or not (control), intended to rupture the endosomes containing the different peptides (N=3). ***, p<0.001;**, p<0.01; n.s. p>0.1 (Student t-test). As negative control, cells are incubated with DMSO.

FIG. 11:

(A) Representation of dTAT-synthetic peptide (Nrh 1-23 C20A and Nrh 1-23 C20A Y16F) whose sequences are represented by SEQ ID NO. 27 and NO. 28. Both dTAT peptides have a TMR (tetramethylrhodamine) fluorescent group grafted to the side chain of the Lysine in position number 2 for tracking purpose.

(B) The interaction between Flag-Nrh and HA-hBD IP3R1 was assessed by co-immunoprecipitation in HeLa cells transfected with a plasmid coding for Flag-Nrh or HA-hBD IP₃R1, pre-incubated with 10 μM control peptide (dTAT 1-23 C20A Y16F) or 10 μM peptide (dTAT 1-23 C20A). In this experiment, dTAT-Nrh 1-23 C20A but not dTAT 1-23 C20A Y16F disrupts the Nrh/IP3R1 complex.

FIG. 12:

(A) MDA-MB-231 cells have been injected into the mammary fat pad of SCID mice. When the tumors reached a mean volume of about 90 mm³, mice were treated with dTAT 1-23 C20A peptide at 10 mg/kg, peritumoral injection, once every three days, or vehicle alone (PBS) with the same injection periodicity. Tumor volume was measured twice a week using calipers (mean±SEM; n=9 mice per condition; **, P<0.01), showing a significant effect of dTAT 1-23 C20A peptide in tumoral growth.

(B) Measurement of the weight of mice over the course of the experiments shown in (A) (mean±SEM; n=9 mice per condition).

FIG. 13: Diagram of calcium influx and apoptosis controlled by Bcl-2 L10/Nrh.

Nrh is able to prevent cytotoxic calcium transfer from ER to mitochondria through the IP3 receptors that would otherwise trigger cell death at the mitochondria. Nrh 1-23 peptide is able to interact directly with Nrh, and inhibit its cytoprotective activity, thus sensitizing Nrh-expressing tumor cells to cell death.

### DETAILED DESCRIPTION OF THE INVENTION

The present invention relates to a competitive inhibitor of the binding of the protein Bcl-2 L10 to the IP3 ligand binding domain (‘BD domain’) of at least one of the IP3R receptors, for its use in the treatment of cancers, wherein the cancer cells express the protein Bcl-2 L10.

FIG. 13 diagrammatically presents the role of the protein Bcl-2 L10/Nrh in processes of cytosolic influx of calcium and in apoptosis.

In the absence of Bcl-2 L10/Nrh, an increase in the local cytosolic calcium quantity leads to the import of calcium into the mitochondria via VDAC (Voltage-Dependent Anion Channel) and MCU (Mitochondrial Calcium Uniporter) channels. In the case of excess calcium, the outer mitochondrial membrane may rupture, and the release of cytochrome c is then observed. The cytochrome c triggers the activation of the Apaf-1/Caspase-9 complex which will lead irreversibly to cell death by apoptosis.

In tumour cells subjected to a treatment intended to induce the apoptosis of said cells, and when the protein Bcl-2 L10 is present, its BH4 domain interacts with the receptor IP3R1 to negatively regulate calcium signalling.

In the presence of a competitive inhibitor which destabilizes the interaction between Bcl-2 L10 and IP3R, hereafter the denoted “peptide Nrh 1-23”, the protein Bcl-2 L10 can no longer bind to IP3R1 and thus the quantity of local cytosolic calcium is re-established and the cell can respond normally to treatment, and thus begin the process of apoptosis.

IP3R Receptors

Three types of IP3R receptors have been described in vertebrates: the protein sequences of each type, IP3R1, IP3R2 and IP3R3, have 60 to 80% identity with their homologues over the total sequence, but this percentage identity is much higher in specific regions such as the binding pocket for the ligand IP3, and the domain defining the channel for the passage of calcium ions.

The Genbank database lists the following sequences identified in humans:
Inositol 1,4,5-triphosphate type 1 receptor/isoform 1, accession number: NP_001093422.2, 2710 amino acids;
Inositol 1,4,5-triphosphate type 1 receptor/isoform 2, accession number: NP_002213.5, 2695 amino acids;
Inositol 1,4,5-triphosphate type 1 receptor/isoform 3, accession number: NP 001161744.1, 2743 amino acids;
Inositol 1,4,5-triphosphate type 2 receptor, accession number: NP 002214.2, 2701 amino acids;
Inositol 1,4,5-triphosphate type 3 receptor, accession number: NP 002215.2, 2671 amino acids.

Although the three types have variable affinities for the ligand IP3, they have the same biological function and are essentially distinguished by differing tissue expression (Mikoshiba, 2007).

In the present invention, the general term “IP3R” is used to denote at least one of the types IP3R1, IP3R2 and IP3R3. Thus, the expressions “binding to at least one of the types of IP3R receptor”, binding to the IP3R receptor” and “binding to one of the IP3R receptors” are used indiscriminately and denote the same interaction.

In a specific embodiment of the invention, the competitive inhibitor of the binding of the protein Bcl-2 L10 to at least one of the IP3R receptors acts exclusively on the IP3 ligand binding domain of IP3R1 and/or IP3R3.

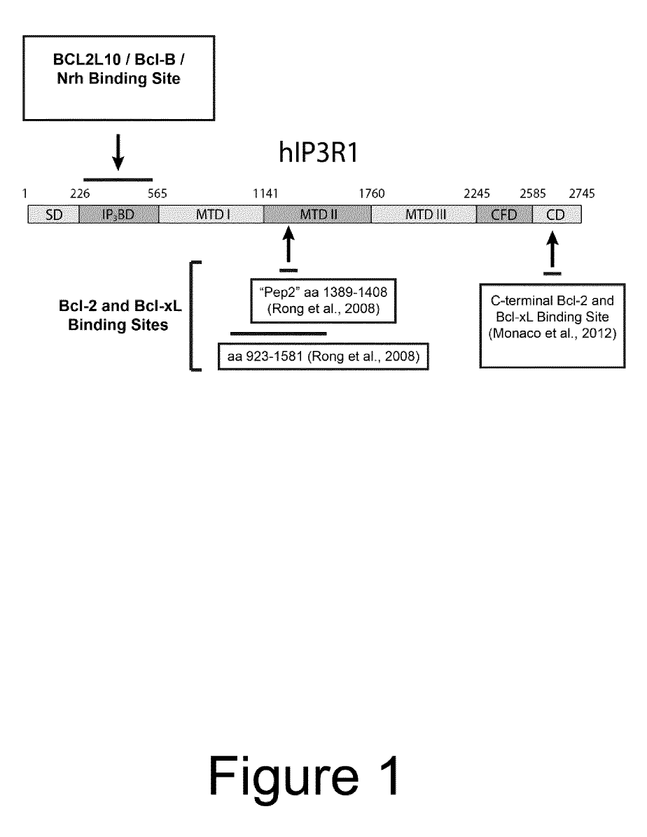

According to a particular embodiment of the invention, the type of IP3R receptor whose binding to Bcl-2 L10 is inhibited is a human protein, and more particularly is one of the isoforms of the protein hIP3R1, the structure of which is represented in FIG. 1.

The IP3 ligand binding domain has been extensively studied. It is also referred to as “BD domain” for “binding domain” and is represented in FIG. 1. It is situated in the N-terminal region and consists of a portion extending from residue 226 to residue 565 in the human protein (Yoshikawa et al., 1996).

Within the context of the invention, the term “competitive inhibitor of the binding of the protein Bcl-2 L10 to the IP3 ligand binding domain of at least one of the types of IP3R receptors” is intended to mean a compound capable of completely or partially inhibiting or destabilizing this binding between Bcl-2 L10 and at least one of the types of IP3R receptors.

Said inhibitor might be able to bind Bcl-2 L10 and/or the domain BD of at least one of the IP3Rs.

The expressions “competitive inhibitor of the binding of the protein Bcl-2 L10 to the IP3R receptor”, “competitive inhibitor of the Bcl-2 L10/IP3R receptor binding” and “inhibitor according to the invention” are used in the present application to denote the competitive inhibitor of the binding of the protein Bcl-2 L10 to the IP3 ligand binding domain of at least one of the types of IP3R receptors.

The term “competitive inhibitor” denotes the fact that the inhibitor is either:
able to bind to the IP3 ligand binding domain, that is to say the BD domain of IP3R (see FIG. 1) and thereby to enter into competition with the protein Bcl-2 L10, the BH4 domain of which interacts with this zone of the IP3R receptor, thereby inhibiting the Bcl-2 L10/IP3R binding; or
able to bind to the protein Bcl-2 L10, and thereby to enter into competition with the BD domain of IP3R, or to inhibit the dimerization of the protein Bcl-2 L10.

Bcl-2 L10/IP3R binding may be quantified according to various techniques well known to those skilled in the art, and especially by immunoprecipitation experiments with specific antibodies, according to the technique described in Example 4 (FIG. 5B).

This Bcl-2 L10/IP3R binding may also be quantified by experiments referred to as “TR-FRET” (time-resolved fluorescence resonance energy transfer), by fluorescence polarization experiments, by SPR (surface plasmon resonance) or by BLI (biolayer interferometry).

The inventors have also shown that this Bcl-2 L10/IP3R binding may be quantified in situ on fixed cells or tissues by means of the PLA technique (proximity ligation assay, FIG. 10A).

The inhibition of the Bcl-2 L10/IP3R binding is characterized by a quantitative reduction in said Bcl-2 L10/IP3R binding.

According to a particular aspect of the invention, the reduction in the binding of Bcl-2 L10 with one of the IP3R receptors is at least 30% relative to the numerical binding value usually observed in the cell in question. In particular, this reduction in the Bcl-2 L10/IP3R binding may be at least 40%, 50%, 60%, 70%, 80%, 90%, 95%, or even 100%, that is to say that no more interaction between Bcl-2 L10 and IP3R is observed at all.

According to a particular aspect of the invention, the competitive inhibitor of the binding of the protein Bcl-2 L10 to the ligand binding domain of at least one of the IP3R receptors is characterized by its function which corresponds to a quantitative inhibition of said Bcl-2 L10/IP3R binding of at least 60%, or even at least 80%, for example as evaluated according to the method described in Example 4.

Bcl-2 L10 Protein

Within the meaning of the invention, the term “Bcl-2 L10 protein” denotes any protein that has been identified as being an isoform or homologue of the human “Bcl-2-like 10” protein, registered in GenBank under the accession number AAG00503.1 and having a sequence of 204 amino acids. This designation, Bcl-2 L10, is used in the present application indiscriminately to the other designations for the same protein, Bcl2-B or Nrh.

The sequence of the protein Bcl-2 L10 is well conserved between mammalian species. For simplification purpose, amino acid numeration starts at the second methionine residue, hence in position 10 on the registered sequence number AAG00503 in GenBank, corresponding to the most-expressed isoform of the Bcl-2 L10 gene. Alignment of the BH4 domains of the zebrafish protein and the human protein have especially made it possible to determine that the arginine at position 6 and the tyrosine at position 16 are conserved (see FIG. 8).

As indicated above, this protein has numerous roles, some of which are still not understood. Thus, a therapeutic strategy which aims to totally eliminate expression of this protein would potentially be dangerous in light of the potential negative effects which may result from the total absence of expression of this protein.

The present invention relates specifically to a competitive inhibitor which prevents binding of the Bcl-2 L10 protein to the IP3R receptor, and which only blocks the effects of Bcl-2 L10 on this receptor.

Competitive Inhibitor Comprising a Peptide Domain

A competitive inhibitor intended to destabilize the Bcl-2 L10/IP3R bond, for its use in the treatment of cancer whose cells express the protein Bcl-2 L10, may be in several forms.

In particular, said inhibitor is in the form of a protein molecule, especially a chimeric peptide, or in the form of a nucleic acid, such as a nucleic aptamer, binding specifically (i) to the BD domain of at least one of the types of IP3R or (ii) to Bcl-2 L10.

In particular, this competitive inhibitor of the binding of the protein Bcl-2 L10 to the IP3R receptor for its use according to the invention comprises a peptide domain.

More particularly, the competitive inhibitor of the binding of the protein Bcl-2 L10 to the IP3R receptor for its use according to the invention comprises a peptide domain, the sequence of which has at least 80% identity with the sequence SEQ ID NO. 1 [RERTELLLADY], the underlined arginine and tyrosine residues being conserved.

The inventors have demonstrated that this inhibitor makes it possible to specifically inhibit the effects of Bcl-2 L10 on the opening of the IP3R calcium channel, and thus to remove the inhibition of the release of calcium into the cytosol.

Within the meaning of the invention, the expression “the sequence has at least 80% identity with the sequence SEQ ID NO. 1” indicates that the sequence of the peptide domain has at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid identity with said reference sequence.

In order to determine the percentage identity of two amino acid sequences, the sequences will be aligned in order to enable optimal comparison. Spaces (gap) may be introduced into one or the other of the sequences to be aligned, in order to enable optimal alignment. The percentage identity of the two amino acid sequences compared may be obtained as described in the book by D. Voet and J. G. Voet, Biochimie [Biochemistry] (2nd Edition, De Boeck & Larcier, 2005, section 7.4, paragraph B).

This identity may be determined by means of comparison algorithms such as Needleman-Wunsch's or Smith-Waterman's global alignment algorithm. The alignments may be carried out especially by the Clustal W or BLAST P software, according to the default parameters or adapted by those skilled in the art.

In a specific embodiment, the competitive inhibitor for its use according to the invention comprises a peptide domain, the sequence of which has at least 80% identity with the sequence SEQ ID NO. 2 [MADPLRERTELLLADYLGYCARE] or with the sequence SEQ ID NO. 3 [ADPLRERTELLLADYLGYCARE], the underlined arginine and tyrosine residues being conserved.

Within the meaning of the invention, the expression “the sequence has at least 80% identity with the sequence SEQ ID NO. 2 or 3” indicates that the peptide domain sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid identity with said reference sequence.

In the examples of the present application, a peptide consisting of this sequence SEQ ID NO. 2 is denoted by the term “Nrh 1-23” and corresponds to the BH4 domain of the human Bcl-2 L10 protein.

This term “Nrh 1-23” may also denote a peptide consisting of the sequence SEQ ID NO. 3, identical to SEQ ID NO. 2 except for the methionine in the first position, which is optional, as is well known by those skilled in the art. This is because the presence or absence of the methionine as first amino acid does not modify the function of the peptides.

Various peptide constructions were produced in order to determine the amino acids essential for the competitive binding of this inhibitor to the BD domain of the IP3R receptor.

The various sequences studied in the examples of the present application are listed in Table 1 below:

TABLE 1

| SEQ | | | |
|---|---|---|---|
| number | Description | Sequence | Figures |
| SEQ ID | Domain of minimal binding | RERTELLLADY | |
| NO. 1 | to IP3Rs | | |
| SEQ ID | BH4 domain of Bc12 L10, | MADPLRERTELLLADYLGYCARE | 4, 5, 6, 7 |
| NO. 2 | enabling binding to IP3R | | |
| | (Nrh 1-23) | | |
| SEQ ID | Domain of binding to IP3Rs | ADPLRERTELLLADYLGYCARE | 4, 5, 6, 7 |
| NO. 3 | without methionine | | |
| SEQ ID | TAT-spacer-domain of | RKKRRQRRRGGSGGADPLRERTE | 9, 10 |
| NO. 4 | binding to IP3Rs without | LLLADYLGYCARE | |
| | methionine | | |
| SEQ ID | Mute binding domain, non- | MADPLAERTELLLADYLGYCARE | 5 |
| NO. 5 | functional “Nrh 1-23 R6A” | | |
| SEQ ID | Mute binding domain, non- | MADPLRERTELLLADFLGYCARE | 5, 6, 7,9, |
| NO. 6 | functional “Nrh 1-23 Y16F” | | 10 |
| SEQ ID | Mute binding domain, | MADPLRERTELLLAAYLGYCARE | 5 |
| NO. 7 | functional “Nrh 1-23 D15A” | | |
| SEQ ID | Mute binding domain, | MADPLRERTELLLADYLGYAARE | 5, 11 |
| NO. 8 | functional “Nrh 1-23 C20A” | | |
| SEQ ID | dTAT Nrh 1-23 C20A, | CKRKKRRQRRRGGSGGADPLRERT | 11, 12 |
| NO. 27 | functional | ELLLADYLGYAARE | |
| SEQ ID | dTAT Nrh 1-23 Y16F, C20A, | CKRKKRRQRRRGGSGGADPLRERT | 11 |
| NO. 28 | non functional | ELLLADFLGYAARE | |

Thus, the competitive inhibitor for its use according to the invention preferentially comprises a peptide domain, the sequence of which has at least 80% identity, and preferentially at least 90% identity, with the sequence SEQ ID NO. 1 or with the sequence SEQ ID NO. 2 or with the sequence SEQ ID NO. 3 or with the sequence SEQ ID NO. 8, the underlined arginine and tyrosine residues being conserved.

According to one embodiment of the invention, the competitive inhibitor comprises such a peptide domain, the sequence of which, having the required percentage identity, consists of 10 to 50 amino acids, preferentially 11 to 40 amino acids, more preferentially 20 to 30 amino acids, and most preferably has a length of between 22 and 28 amino acids, the limit values indicated above being included in the range.

According to one embodiment of the invention, the competitive inhibitor comprises a peptide domain that contains 50 amino acids or less.

Several sequences for such a peptide domain, having at least 80% identity with the sequence shown in SEQ ID NO. 1, may be envisaged. Indeed, for two peptide domains of a length of 11 amino acids, as soon as 9 amino acids are identical across the two sequences, the percentage identity between these two peptide sequences is 81.81% identity.

Mention may be made of the examples as in Table 2 below, this list of course being non-limiting, of peptide domains having at least 80% identity with the sequence SEQ ID NO. 1, in which X represents any amino acid chosen from the twenty known amino acids:

TABLE 2

| SEQ number | Sequence |
|---|---|
| SEQ ID NO. 9 | RERTELLXXDY |
| SEQ ID NO. 10 | RERTXXLLADY |
| SEQ ID NO. 11 | REXTELLLAXY |

According to a particular aspect of the invention, said competitive inhibitor for its use in the treatment of cancers whose cells express Bcl-2 L10 comprises a peptide domain, the sequence of which consists of a sequence chosen from one of the sequences SEQ ID NO. 1, SEQ ID NO. 2, SEQ ID NO. 3, SEQ ID NO. 7, SEQ ID NO. 8, SEQ ID NO. 9, SEQ ID NO. 10 or SEQ ID NO. 11.

According to another embodiment of the invention, said competitive inhibitor for its use in the treatment of cancers whose cells express Bcl-2 L10 consists in a peptide domain, the sequence of which consists of one of the sequences SEQ ID NO. 1, SEQ ID NO. 2, SEQ ID NO. 3, SEQ ID NO. 7, SEQ ID NO. 8, SEQ ID NO. 9, SEQ ID NO. 10 or SEQ ID NO. 11.

According to this embodiment, such a competitive inhibitor, in the form of a peptide, will be able to be produced directly by chemical synthesis, or synthesized within the cell by virtue of the cell machinery from a plasmid or a vector comprising a nucleic acid sequence coding for one of the peptide sequences mentioned above. Said plasmid or vector will have been introduced beforehand into the cell by a conventional transfection, injection or viral infection technique. Advantageously the peptide synthesized within the cell will be fused with a particular targeting sequence, as is presented below.

Homodimerisation of Bcl-2 L10

The inventors have observed that Bcl-2 L10 homodimerisation can be prevented by incubation with the Nrh 1-23 peptide (SEQ ID NO. 2 or 3). This result is presented in example 5 and FIG. 7.

According to a specific embodiment, an inhibitor for its use according to the invention further inhibits the homodimerisation of Bcl-2 L10.

According to another embodiment, the invention relates to an inhibitor of Bcl-2 L10 homodimerisation, for its use in the treatment of cancers whose cells express the protein Bcl-2 L10.

In order to identify compounds able to inhibit the dimerization of Bcl-2 L10, the man skilled in the art could use the experiments such as described in example 5.

Said compounds able to inhibit the dimerization of Bcl-2 L10 are preferentially selected among the compounds described in the present application, in particular those comprising a peptide domain, preferentially comprising a peptide domain the sequence of which has at least 80% identity with the sequence SEQ ID NO. 1 [RERTELLLADY], the underlined arginine and tyrosine residues being conserved.

According to a particular aspect of the invention, said Bcl-2 L10 homodimerisation inhibitor for its use in the treatment of cancers whose cells express Bcl-2 L10 comprises a peptide domain, the sequence of which consists of a sequence chosen from one of the sequences SEQ ID NO. 1, SEQ ID NO. 2, SEQ ID NO. 3, SEQ ID NO. 7, SEQ ID NO. 8, SEQ ID NO. 9, SEQ ID NO. 10 or SEQ ID NO. 11.

According to another embodiment of the invention, said Bcl-2 L10 homodimerisation inhibitor for its use in the treatment of cancers whose cells express Bcl-2 L10 consists in a peptide domain, the sequence of which consists of one of the sequences SEQ ID NO. 1, SEQ ID NO. 2, SEQ ID NO. 3, SEQ ID NO. 7, SEQ ID NO. 8, SEQ ID NO. 9, SEQ ID NO. 10 or SEQ ID NO. 11.

Structure of the Inhibitor to Facilitate Passage Thereof Through Cell Membranes

As set out above, the IP3R receptor is located in the membrane of the endoplasmic reticulum. Thus, a competitive inhibitor must be able to reach said membrane in order to bind to the IP3R receptor.

It is therefore important that said competitive inhibitor, for its use in the treatment of cancers whose cells express the protein Bcl-2 L10, when it is administered to a person, for example through the blood, may cross the cell membranes and thus be present in the cytosol where it may become attached to the IP3R receptor. Cell membranes are impermeable to hydrophilic molecules such as peptides.

The problem of the entry of peptides into living cells, and their access to the various intracellular compartments, in particular the cytoplasmic compartment, is of great importance for their therapeutic use.

In order to facilitate the transport of a peptide through a biological membrane, three techniques are mainly used:
the use of a compound administered at the same time as the peptide, the presence of which facilitates the transport of a peptide through cell membranes; or else
the use of structures for encapsulation of the peptide, such as liposomes or polymers; or else
the use of a targeting compound, fused at the N terminal or C terminal of the peptide, to facilitate its passage through cell membranes and/or to direct it towards specific cell structures.

These three techniques may be used independently of one another, or be used simultaneously.

The use of encapsulation structures, enabling both the protection of the peptides from enzymatic degradation, and the passage of the hydrophilic peptides through the hydrophobic barrier, is well known to those skilled in the art.

Liposomes are vesicular structures composed of lipid layers, making it possible to encapsulate hydrophilic compounds within lipids. Liposomes are increasingly used in human therapy, as medicament vectors.

The most commonly used polymers for the encapsulation of hydrophilic active compounds (peptides) are derivatives of lactic and glycolic acids (PLGA, PLA), ethyl cellulose, and poly(epsilon-caprolactone), which are biodegradable.

Targeting Compounds

A targeting compound, hereinafter denoted [ADR], fused to a peptide domain having a function of competitive inhibitor, may be a peptide or non-peptide molecule.

The targeting compound fused to the peptide of interest may be in particular any amino acid sequence facilitating and/or mediating the transport of said peptide from the exterior of a cell to its interior. Such sequences are known to those skilled in the art. They generally consist of 2 to 20 amino acids. Said sequence enabling the penetration of said peptide into a cell may be chosen depending on the cell type of said cell, in order to optimize penetration efficacy.

Among these targeting compounds, mention may especially be made of the peptide derived from the TAT protein of the retrovirus HIV, with the sequence as shown in SEQ ID NO. 12 (RKKRRQRRR), the penetratin consisting of a sequence as shown in SEQ ID NO. 13 (RQIKIWFQNRRMKWKK), the sequence referred to as “X7/11R sequence”, denoting any peptide sequence of 7 to 20 amino acids containing between seven and eleven arginine residues (7/11R), in which the arginine residues may be placed randomly within said sequence, and the homeodomain type peptides.

Mention may also be made of specific targeting compounds, such as the mitochondrial targeting domain ActA of L. monocytogene, or the endoplasmic reticulum targeting domain cb5, derived from the human Cytochrome b5 protein.

According to a preferred embodiment, the targeting compound enabling the penetration of said competitive inhibitor into a cell is the peptide derived from HIV-TAT of sequence SEQ ID NO. 12.

According to a particular embodiment of the invention, the HIV-TAT targeting domain fused to the peptide may be dimerized, to increase the bioavailability of the peptide within the cell, as is presented in international application WO 2015/038662.

According to another particular embodiment of the invention, the HIV-TAT targeting domain may be preceded with the two amino acids ‘CK’, i.e. a cysteine and a lysine, whose presence increases the dimerization of the HIV-TAT domain in oxidative conditions.

Dimeric TAT has enhanced endosomal escape properties that allow an efficient cell entry of the peptides, without the use of an external physical agent such as light.

### Structure of the Competitive Inhibitor of the Invention

According to the invention, the competitive inhibitor for its use in the treatment of cancers, the cells of which express Bcl-2 L10, may be composed of several peptide or non-peptide domains, coupled to one another.

In particular, the competitive inhibitor is constructed according to the following structure: [ADR]ₓ-[ESP]_{y}-[DOM]-[SADR]_{z} in which
ADR denotes a targeting compound,
ESP denotes a spacer,
DOM is a peptide domain as defined above,
and SADR denotes a specific intracellular targeting domain,
   and in which x, y and z are equal to 0 or 1 independently of one another, and the sum (x+y+z) is equal or superior to 1.

The targeting compound will in particular be one of the compounds mentioned above. In an embodiment, the targeting compound is the HIV-TAT targeting domain preceded with the two amino acids ‘CK’

In order to obtain an efficient dimerization of the peptide, it is preferable that the sequence of the peptide does not comprise any cysteine residue.

Therefore, according to a preferred embodiment of the invention, when the HIV-TAT targeting domain is preceded with the two amino acids ‘CK’, the peptide domain DOM does not comprise any cysteine residue.

In a more preferred embodiment of the invention, when the HIV-TAT targeting domain is preceded with the two amino acids ‘CK’, the peptide domain DOM comprises the sequence SEQ ID NO. 8.

A spacer, also referred to as a molecular spacer, denotes a segment ensuring binding between two molecule portions. Within the meaning of the present invention, this is a domain intended to separate the active peptide domain and the targeting sequence, which due to its hydrophobicity could adversely affect the function of the active peptide domain.

This spacer, also referred to as “linker” or “linking moiety”, may be a peptide or non-peptide molecule. If it is a peptide, it is at least 3 amino acids long and at most 50 amino acids long in terms of size. The size of the spacer peptide is preferably between 5 and 30 amino acids long and most preferably between 5 and 20 amino acids long.

Spacers are generally classified in three categories: flexible spacers, rigid spacers and in vivo-cleavable spacers. All the spacers listed in the review by (Chen et al., 2013) are able to be used in the structure of the competitive inhibitor according to the invention.

This spacer, located between the active peptide domain and the targeting sequence, is maintained by bonds which must be sufficiently stable to maintain the molecule of overall structure [ADR]ₓ-[ESP]_{y}-[DOM]-[SADR]_{z} throughout the passage through the cytoplasmic membrane and capture by the endosome. Subsequently, the bond may no longer be necessary and the molecule may be cleaved at the spacer.

According to one embodiment of the invention, the spacer belongs to the in vivo-cleavable spacers and the structure [ADR]ₓ-[ESP]_{y}-[DOM]-[SADR]_{z} is cleaved within the cell.

According to another embodiment of the invention, the spacer is of flexible type and is composed of glycine and serine residues.

The examples presented below describe competitive inhibitors of structure [ADR]ₓ-[ESP]_{y}-[DOM]-[SADR]_{z}, in which y=l1, comprising such a flexible spacer of sequence as shown in SEQ ID NO. 14 (GGSGG).

The different sequences mentioned above are presented in Table 3:

TABLE 3

| Sequence | | |
|---|---|---|
| number | Description | Sequence |
| SEQ ID NO. 12 | HIV-TAT | RKKRRQRRR |
| | peptide | |
| SEQ ID NO. 13 | penetratin | RQIKIWFQNRRMKWKK |
| SEQ ID NO. 14 | Spacer | GGSGG |
| SEQ ID NO. 15 | ER-targeting | WWTNWVIPAISAVAVALMYRLYM |
| | domain of | AED |
| | human | |
| | Cytochrome b5 | |
| SEQ ID NO. 16 | “KDEL” ER- | KDEL |
| | targeting | |
| | domain | |
| SEQ ID NO. 17 | “KKMP” ER- | KKMP |
| | targeting | |
| | domain | |

According to a particular aspect of the invention, the amino acid sequence of the spacer is also a marker for detecting or purifying the peptide. For example, the spacer peptide may be composed of the peptide “HA TAG”.

According to a particular aspect of the invention, the competitive inhibitor is constructed according to the following structure: [ADR]ₓ-[ESP]_{y}-[DOM]-[SADR]_{z} in which
ADR denotes a targeting compound,
ESP denotes a spacer,
DOM is a peptide domain having one of the sequences chosen from SEQ ID NO. 1, SEQ ID NO. 2, SEQ ID NO. 3, SEQ ID NO. 4, SEQ ID NO. 7, SEQ ID NO. 8, SEQ ID NO. 9, SEQ ID NO. 10, and SEQ ID NO. 11,
SADR denotes a specific intracellular targeting peptide domain,
and in which x, y and z are equal to 1.

According to a particular aspect of the invention, the competitive inhibitor is constructed according to the following structure: [ADR]ₓ-[ESP]_{y}-[DOM]-[SADR]_{z} in which
ADR denotes a targeting compound,
ESP denotes a spacer,
DOM is a peptide domain having one of the sequences SEQ NO. 2 or NO. 3 or NO. 8,
SADR denotes a specific intracellular targeting peptide domain,
and in which x, y and z are equal to 1.

According to another particular aspect of the invention, the competitive inhibitor is constructed according to the following structure: [ADR]ₓ-[ESP]_{y}-[DOM]-[SADR]_{z} in which ADR denotes a targeting compound consisting of a peptide derived from TAT-HIV, of sequence SEQ ID NO. 12, and in which x is equal to 1.

According to another particular aspect of the invention, the competitive inhibitor is constructed according to the following structure: [ADR]ₓ-[ESP]_{y}-[DOM]-[SADR]_{z} in which ESP denotes a spacer of sequence SEQ ID NO. 14, and in which y is equal to 1.

According to another particular aspect of the invention, the competitive inhibitor is constructed according to the following structure: [ADR]ₓ-[ESP]_{y}-[DOM]-[SADR]_{z} in which ESP denotes a spacer of sequence SEQ ID NO. 14, and in which z is equal to 1.

The competitive inhibitor for its use according to the invention may consist exclusively of peptide domains and may especially be characterized in that the domains ADR, ESP and SADR are peptide domains.

Table 4 below represents specific sequences of peptides for their use according to the invention.

TABLE 4

| | | |
|---|---|---|
| SEQ ID | TAT-spacer-domain of | RKKRRQRRRGGSGGADPLRERTELLLADYLGYC |
| NO. 4 | binding to IP3Rs | ARE |
| | without methionine | |
| SEQ ID | TAT-linker-domain for | RKKRRQRRRGGSGGADPLRERTELLLADYLGYC |
| NO. 18 | binding to IP3Rs | AREWWTNWVIPAISAVAVALMYRLYMAED |
| | without methionine - | |
| | targeting sequence Cb5 | |
| SEQ ID | CK-TAT-spacer- | CKRKKRRQRRRGGSGGADPLRERTELLLADYLG |
| NO. 27 | domain for binding to | YAARE |
| | IP3Rs C20A without | |
| | methionine | |

The competitive inhibitor for its use according to the invention may comprise at least one peptide having the peptide sequence represented in any one SEQ ID NO. 4, 18 or 27.

According to yet another preferred aspect of the invention, the competitive inhibitor for its use according to the invention consists of a peptide having the peptide sequence represented in SEQ ID NO. 4.

According to yet another preferred aspect of the invention, the competitive inhibitor for its use according to the invention consists of a peptide having the peptide sequence represented in SEQ ID NO. 18.

According to yet another preferred aspect of the invention, the competitive inhibitor for its use according to the invention consists of a peptide having the peptide sequence represented in SEQ ID NO. 27.

Generally, the present application relates to a competitive inhibitor of the binding of the protein Bcl-2 L10 to the ligand binding domain of at least one of the IP3R receptors, for use as medicament.

Indeed, the inventors have demonstrated the therapeutic effects of this competitive inhibitor, which prevents binding of the Bcl-2 L10 protein to the IP3R receptor and thus removes the inhibition of the opening of the calcium channel which Bcl-2 L10 imposes on the IP3R receptor.

By rendering the IP3R receptor non-dependent on the presence of Bcl-2 L10, it becomes sensitive to other signals again, and especially to the changes in the concentration of its ligand, IP3.

Thus, the competitive inhibitor may be used as a medicament for all sorts of therapeutic applications, especially in pathological conditions linked directly or indirectly to the functioning and/or to the regulation of the IP3R receptor.

This medicament is mainly intended for humans but may also be used in vertebrate animals, in particular in mammalian animals.

In the use thereof as medicament, it is understood that the amount of competitive inhibitor to be administered may vary as a function of the weight of the patient in question and of the desired effect, and also of the dosage to be adapted depending on the frequency of treatment. These dosages are well known to those skilled in the art and will be able to be readily determined by them using their general knowledge.

A competitive inhibitor for its use according to the invention may be administered via any suitable route, such as orally, sublingually, rectally, parenterally, intraperitoneally, intradermally, transdermally, intratracheally, topically or ophthalmically.

An administration by injection may be carried out, for example, intraperitoneally, intradermally, subcutaneously, intravenously or intramuscularly.

Any mucosal route may also be used, such as the genitourinary, anorectal, respiratory, bucconasal, sublingual routes, or a combination thereof.

The competitive inhibitor according to the invention is used as a medicament in the treatment of cancers, the cells of which express the protein Bcl-2 L10.

“Treating cancer” is intended to mean causing all the cancerous cells, and in particular the tumour of origin, to decrease or even disappear completely from the patient's body.

Within the meaning of the invention, the expression “cancer, the cells of which express the protein Bcl-2 L10” covers numerous types of cancer and especially breast, prostate and lung cancers and haematopoietic tumours such as acute myeloid leukaemias and myelodysplastic syndromes.

According to a particular aspect of the invention, the competitive inhibitor for its use in the treatment of cancers, the cells of which express the protein Bcl-2 L10, is administered in combination with at least a second active agent and/or any conventional method for treating cancer.

In particular, said second active agent is a chemotherapy product or immunotherapy product.

In one embodiment, said conventional method for treating cancer is surgery or radiotherapy.

Within the meaning of the invention, “chemotherapy product” is intended to mean a therapeutic compound used in the treatment of cancer, intended to induce apoptosis of cancer cells. Such products are well known to those skilled in the art.

According to a particular aspect of the invention, said chemotherapy product will be chosen from the list comprising the products referred to as cisplatin, carboplatin, oxaliplatine, bendamustine, dacarbazine, temozolomide, estramustine, methotrexate, azacitidine, capecitabine, cytarabine, fluorouracil, gemcitabine, tegafur, pemetrexed, nelarabine, hydroxycarbamide, raltitrexed, doxorubicin, epirubicin, mitoxantrone, daunorubicin, idarubicin, bleomycin, mitomycin, dactinomycin, irinotecan, topotecan, etoposide, docetaxel, paclitaxel, thapsigargin, and derivatives thereof.

Within the meaning of the invention, the expression “administered in combination with at least one chemotherapy product” indicates that the two therapeutic compounds, the competitive inhibitor and the chemotherapy product, may be used concomitantly or sequentially, and in particular they may be administered separately in time, over the course of the treatment of a patient.

The invention also relates to a chemotherapy product, for use thereof in combination with at least one competitive inhibitor as described above in the treatment of cancers, the cells of which express the protein Bcl-2 L10.

The invention also relates to a competitive inhibitor used as medicament, in combination with at least one chemotherapy product, said competitive inhibitor being a modulator of the response of the cancers, the cells of which express the protein Bcl-2 L10, to chemotherapy treatments.

The term “response modulator” denotes the ability of the competitive inhibitor according to the invention to optimize the therapeutic effects of the chemotherapy product(s), especially of the products used for the treatment of cancers, the cells of which express the protein Bcl-2 L10. Thus, when the competitive inhibitor is used as medicament, in combination with at least one chemotherapy product, in the context of the treatment of a cancer, the cells of which express the protein Bcl-2 L10, said treatment with the chemotherapy product will be more effective against said cancer.

The expression “effective against said cancer” denotes especially the fact that the amounts of chemotherapy product used for the treatment will be less than those customarily used, when the competitive inhibitor is used as medicament in combination with said chemotherapy product.

The expression “effective against said cancer” may also denote the fact that the treatment with said chemotherapy product will be shorter than that customarily carried out in a patient suffering from a cancer, the cells of which express the protein Bcl-2 L10, when the competitive inhibitor is used as medicament in combination with said chemotherapy product.

The invention also relates to a process for treating a cancer, the cells of which express the protein Bcl-2 L10, comprising a step of administering a competitive inhibitor of the binding of the protein Bcl-2 L10 to the ligand binding domain of at least one of the IP3R receptors to a patient suffering from said cancer.

The invention also relates to a process for treating a cancer, the cells of which express the protein Bcl-2 L10, comprising a step of administering a competitive inhibitor of the binding of the protein Bcl-2 L10 to the ligand binding domain of at least one of the IP3R receptors, and another step of administering at least one chemotherapy product, to a patient suffering from said cancer, the two administration steps being able to be concomitant or sequential.

Competitive Inhibitor

The present invention also concerns a competitive inhibitor of the binding of the protein Bcl-2 L10 to the ligand binding domain of at least one of the IP3R receptors, constructed according to the following structure: [ADR]ₓ-[ESP]_{y}-[DOM]-[SADR]_{z}, in which
ADR is a targeting compound,
ESP is a spacer,
DOM is a peptide domain, the sequence of which has at least 80% identity with the sequence SEQ ID NO. 1 [RERTELLLADY], the underlined arginine and tyrosine residues being conserved,
SADR denotes a specific intracellular targeting peptide domain,
   wherein x, y and z are equal to 0 or 1 independently of one another, and the sum (x+y+z) is equal or superior to 1.

The present invention concerns all inhibitors described previously, constructed according to the structure above.

In a specific embodiment, DOM is a peptide domain, the sequence of which has at least 80% identity, and preferentially at least 90% identity, with the sequence SEQ ID NO. 1 or with the sequence SEQ ID NO. 2 or with the sequence SEQ ID NO. 3 or with the sequence SEQ ID NO. 8, the underlined arginine and tyrosine residues being conserved.

In an embodiment, the competitive inhibitor is characterized in that the domains ADR, ESP and SADR are peptide domains.

In a specific embodiment, the competitive inhibitor comprises an amino acid sequence represented by an amino acid sequence selected among the group consisting of: SEQ ID NO. 4, SEQ ID NO. 18 and SEQ ID NO. 27.

In a more specific embodiment, the competitive inhibitor consists in a peptide having an amino acid sequence selected among the group consisting of: SEQ ID NO. 4, SEQ ID NO. 18 and SEQ ID NO. 27.

Pharmaceutical Composition

According to another of its aspects, the invention relates to a pharmaceutical composition comprising, in a pharmaceutically acceptable medium, at least one competitive inhibitor as described above.

According to a preferred embodiment, the invention relates to a pharmaceutical composition comprising, in a pharmaceutically acceptable medium, at least one competitive inhibitor of the binding of the protein Bcl-2 L10 to the ligand binding domain of at least one of the IP3R receptors, constructed according to the following structure: [ADR]ₓ-[ESP]_{y}-[DOM]-[SADR]_{z}, in which
ADR is a targeting compound,
ESP is a spacer,
DOM is a peptide domain, the sequence of which has at least 80% identity with the sequence SEQ ID NO. 1 [RERTELLLADY], the underlined arginine and tyrosine residues being conserved,
SADR denotes a specific intracellular targeting peptide domain,

wherein x, y and z are equal to 0 or 1 independently of one another, and the sum (x+y+z) is equal or superior to 1.

Such competitive inhibitor may especially comprise a peptide domain (DOM) of sequence represented in SEQ ID NO. 1, SEQ ID NO. 2, SEQ ID NO. 3, SEQ ID NO. 4, SEQ ID NO. 7, SEQ ID NO. 8, SEQ ID NO. 9, SEQ ID NO. 10, SEQ ID NO. 11.

The competitive inhibitor may also comprise an amino acid sequence represented by an amino acid sequence selected among the group consisting of: SEQ ID NO. 4, SEQ ID NO. 18 and SEQ ID NO. 27.

Such competitive inhibitor may also consist of a peptide domain of sequence as represented in one of the sequences SEQ ID NO. 4, SEQ ID NO. 18 and SEQ ID NO. 27.

A competitive inhibitor of the Bcl-2 L10/IP3R receptor binding or a pharmaceutical composition of the invention may be formulated with any pharmaceutically acceptable medium or vehicle, and may be formulated in any solid, semi-solid, liquid or gaseous form, such as a tablet, a capsule, a gel capsule, a powder, a granule, an emulsion, a suspension, a gel, a microsphere or an inhaled form.

According to one embodiment, a competitive inhibitor of the Bcl-2 L10/IP3R receptor binding in accordance with the invention or a pharmaceutical composition of the invention may be formulated for the oral route, in the form of a tablet, a capsule or a gel capsule, for prolonged or controlled release, a pill, a powder, a solution, a suspension, a syrup or an emulsion.

According to another embodiment, a competitive inhibitor of the Bcl-2 L10/IP3R receptor binding in accordance with the invention or a pharmaceutical composition of the invention may be prepared in injectable form. A polypeptide or a nucleic acid of the invention may be formulated with different carriers, such as a liposome or a transfection polymer.

A pharmaceutical composition of the invention may preferably comprise an inhibitor as defined in the present description, in suspension in a pharmaceutically acceptable vehicle, for example an aqueous vehicle. Various aqueous vehicles may be used, for example water, a saline buffer solution, a 0.4% or 0.3% glycine solution, or a hyaluronic acid solution.

According to a preferred aspect of the invention, the pharmaceutical composition moreover comprises an agent making it possible to facilitate the transport of the peptides through a biological membrane, such as for example the CHARIOT™ Protein Delivery Reagent.

A pharmaceutical composition according to the invention may be sterilized by any conventional known method, such as filtration. The resulting aqueous solution may be conditioned to be used as is, or be lyophilized. A lyophilized preparation may be combined with a sterile solution before use.

A pharmaceutical composition of the invention may comprise any required pharmaceutically acceptable excipient, such as buffer agents or agents for adjusting the pH or the isotonicity, and wetting agents. A pharmaceutical composition according to the invention may also comprise one or more antioxidants, one or more preservatives and optionally other medicament active agents.

According to a particular embodiment, a pharmaceutical composition of the invention comprises an effective amount of the competitive inhibitor of the Bcl-2 L10/IP3R interaction.

An effective amount of an inhibitor according to the invention is an amount which, alone or in combination with subsequent doses, induces the desired response, namely an inhibitory effect with regard to the Bcl-2 L10/IP3R receptor binding, and consequently the removal of the inhibition that Bcl-2 L10 imposes on the function of the calcium channel of the IP3R receptor. The effective amount of such an inhibitor may depend on one parameter or on a plurality of parameters, such as the route of administration, single or multiple dose administration, or the patient's characteristics, which include age, physical condition, height, weight, and the presence of illnesses or diseases. These parameters and their influences are well known to those skilled in the art and may be determined by any known method.

Said composition will be able to be used in any type of treatment, especially in the context of preventing and/or treating all types of cancers, the cells of which express the protein Bcl-2 L10.

### EXAMPLES

### Example 1. Cellular Localization of the Protein Bcl-2 L10 (Nrh)

Human mammary carcinoma cells MDA-MB-231 endogenously expressing the protein Bcl-2 L10/Nrh are cultured then lysed. Several extracts are deposited on the gel: the total fraction, the mitochondrial fraction (Mito), the reticular fraction (ER) and the soluble fraction obtained after centrifugation at 100000 G (S100). The proteins vinculin, calnexin and FOF1 are used as control.

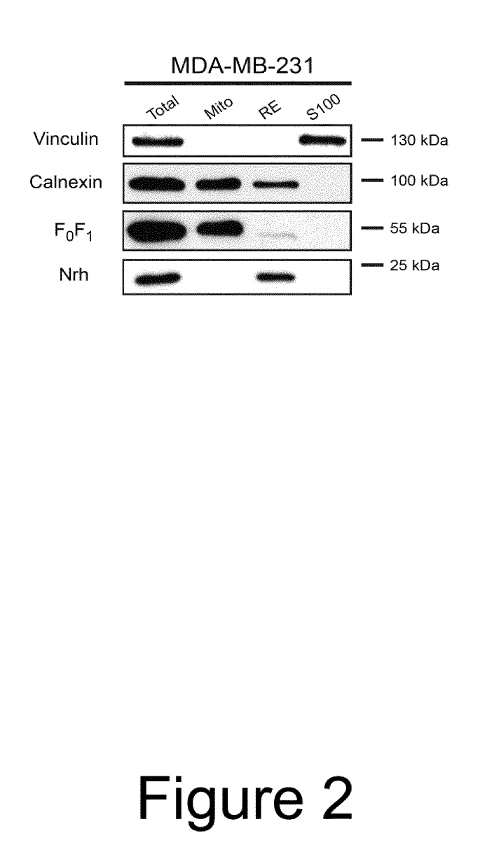

The localization of the protein Bcl-2 L10/Nrh in the endoplasmic reticulum (ER) is clearly visualized in the third column of FIG. 2.

### Example 2. Effects of Interfering RNAs Preventing Expression of the Protein Nrh, in the Presence or Absence of Thapsigargin

MDA-MB-231 cells were transfected with siRNAs directed against the mRNA of Nrh (si3-Nrh or si6-Nrh), or with a control siRNA (siSCR). In these cells, the expression of Nrh is greatly reduced.

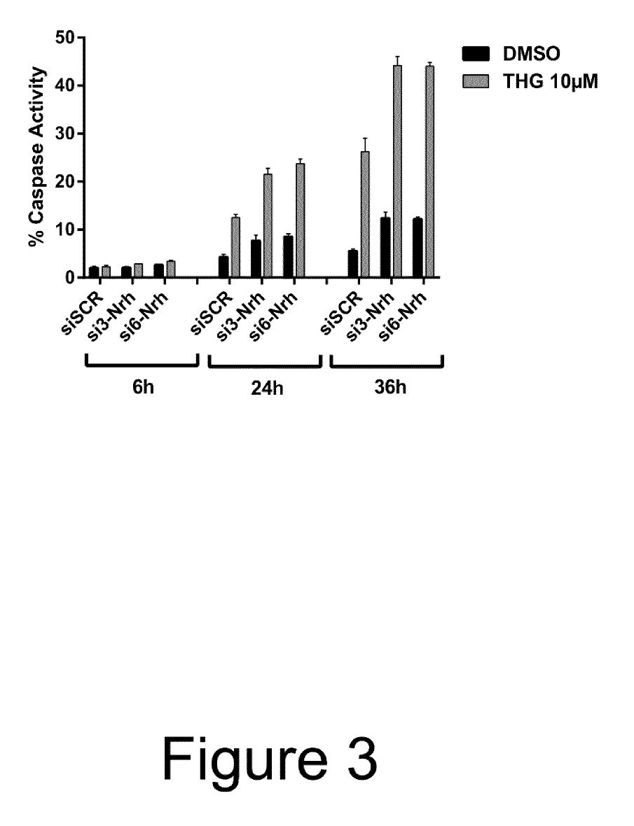

48 hours after transfection, the cells are incubated for 6 hours, 24 hours and 36 hours with DMSO (control) or 10 μM of thapsigargin (THG). The results are presented in FIG. 3. The activity of caspases 3 and 7 is measured in percentages. Activity of greater than 10% indicates that the cells have entered into the process of apoptosis, which is expected in the presence of thapsigargin, a chemotherapy compound that induces cell apoptosis. On the other hand, in the presence of DMSO, the cells should not exhibit caspase activity.

However, in the presence of the interfering RNAs blocking the expression of Bcl-2 L10/Nrh, even without thapsigargin treatment, the cells incubated for 36 h have caspase activity of greater than 10%.

Moreover, in the presence of thapsigargin, the cells transfected with the interfering RNAs have much greater caspase activity than that observed for the cells transfected with the control interfering RNA.

Thus, the absence of expression of the protein Bcl-2 L10/Nrh leads to a significant potentiation of the effects of thapsigargin, this effect being increasingly pronounced over the course of the incubation kinetics.

### Example 3. Effects of Mutant or Fusion Proteins, Derived from Bcl-2 L10/Nrh, in the Presence or Absence of Thapsigargin

Several polypeptide constructs were produced starting from the gene nrh:
the human Bcl-2 L10/Nrh protein in its wild-type, whole form: Nrh-WT;
the protein Bcl-2 L10/Nrh in its wild-type, whole form with a compound for targeting the endoplasmic reticulum derived from the human Cytochrome b5 protein: Nrh-cb5: Nrh-cb5,
the protein Bcl-2 L10/Nrh in its wild-type, whole form with a compound for targeting the mitochondrion ActA of L. monocytogene: Nrh-ActA,
the protein Bcl-2 L10/Nrh, from which the membrane-anchoring domain has been deleted: Nrh-dTM,
the protein Bcl-2 L10/Nrh, from which the N-terminal BH4 domain has been deleted: Nrh-dBH4.

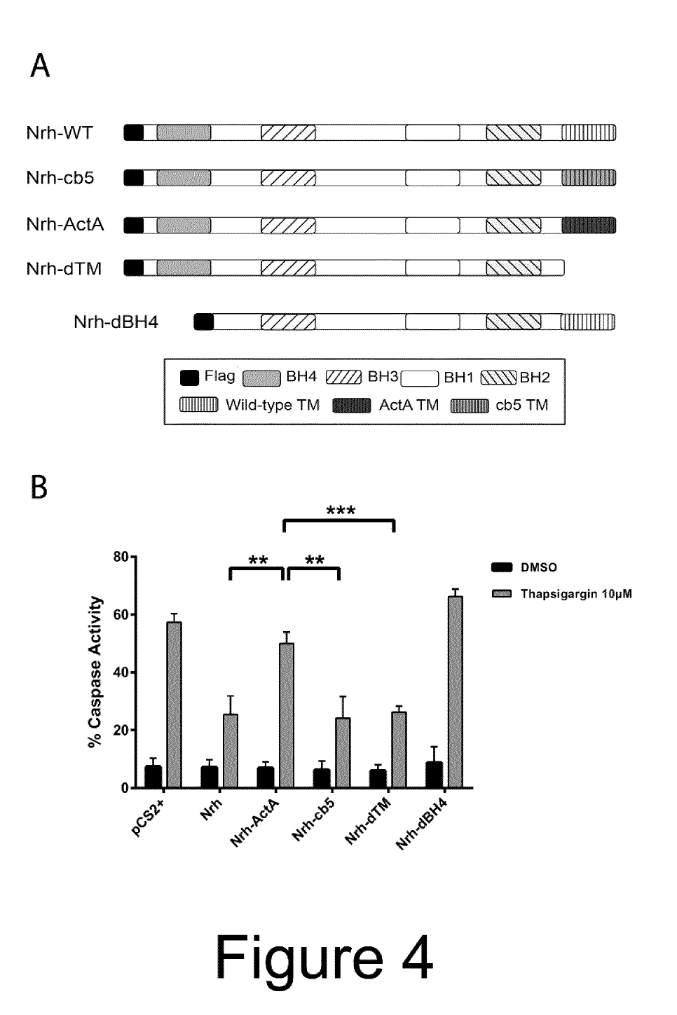

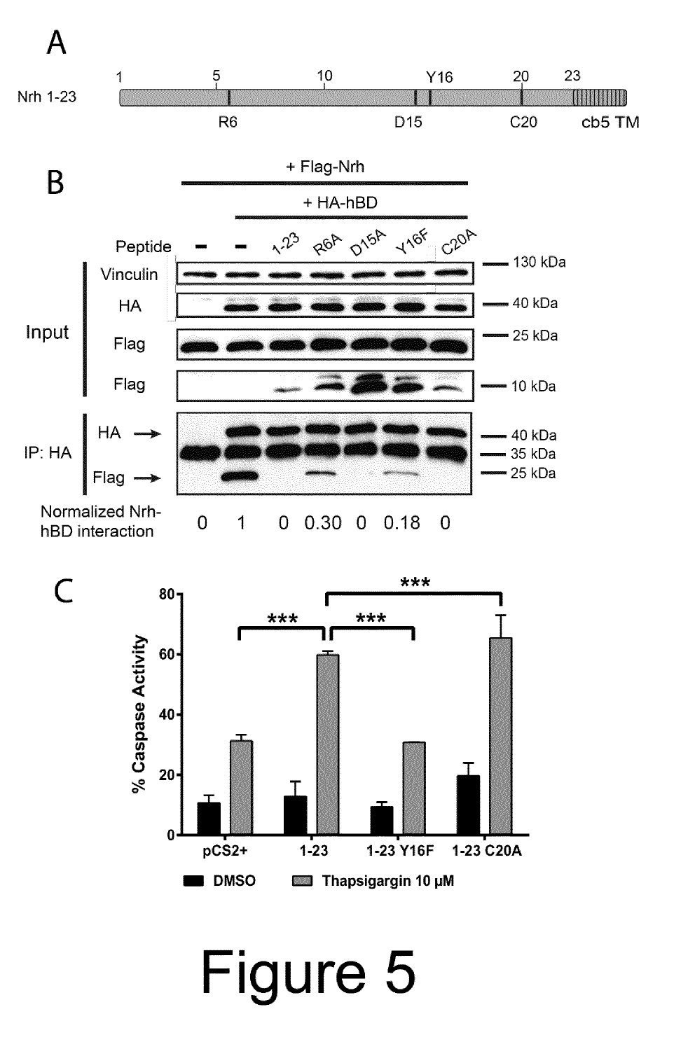

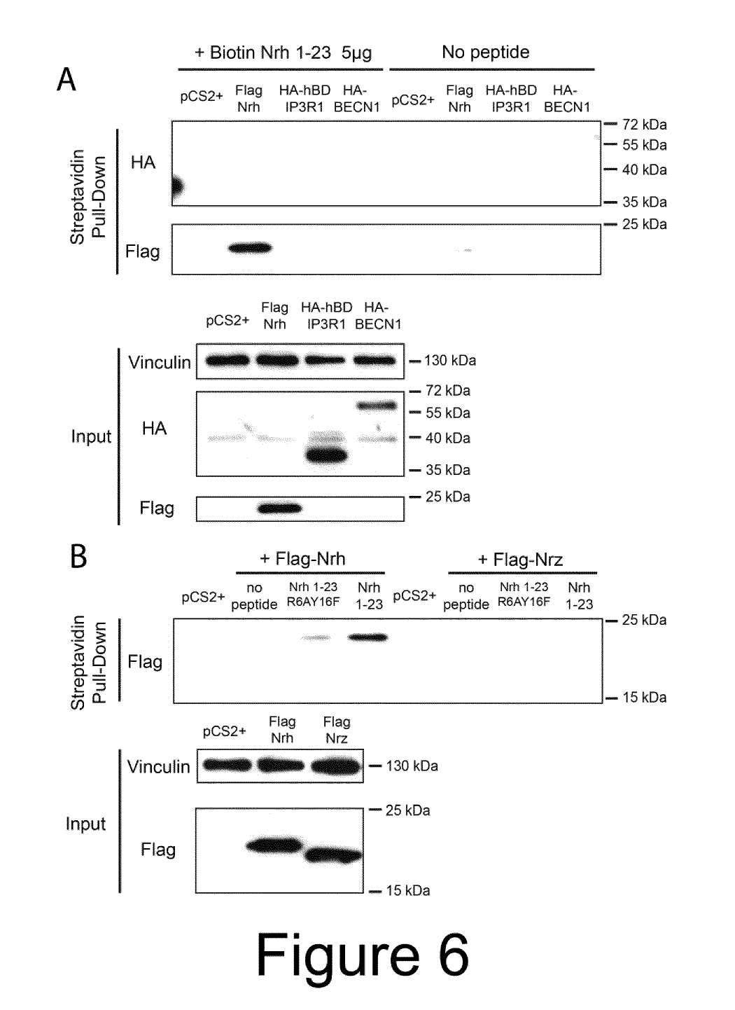

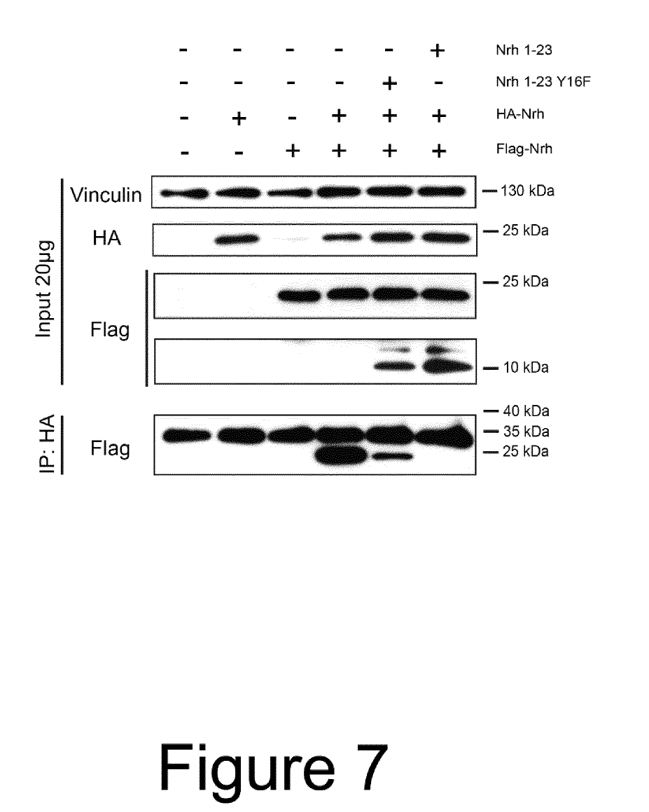

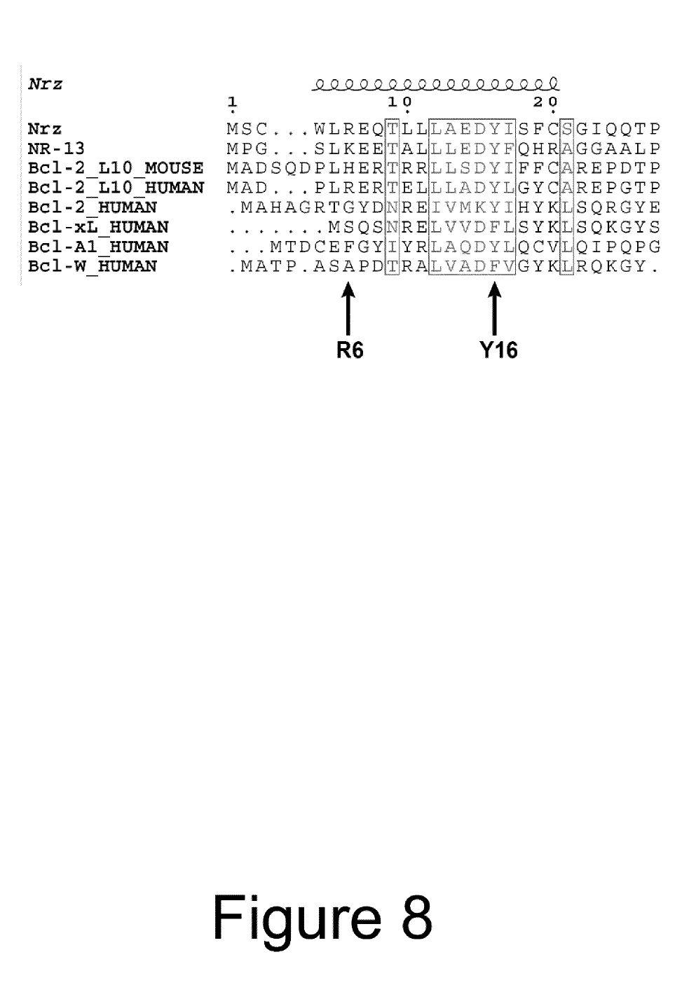

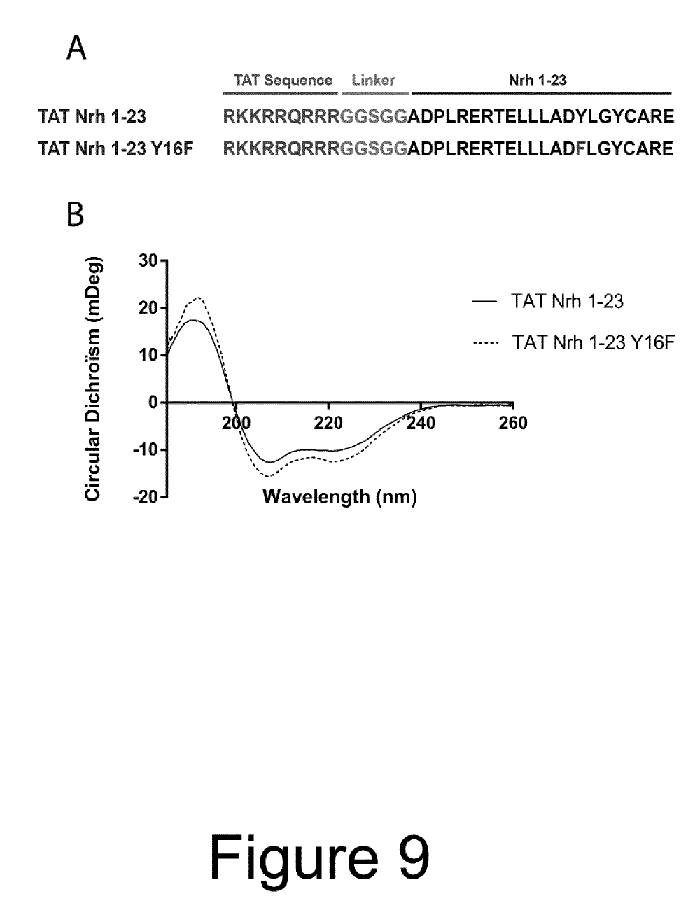

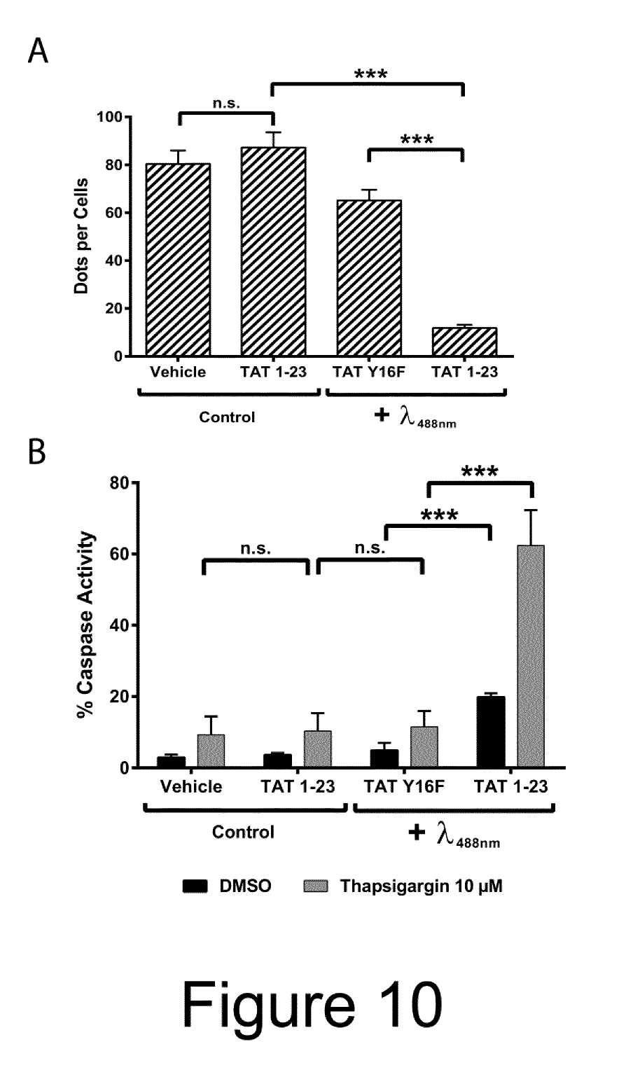

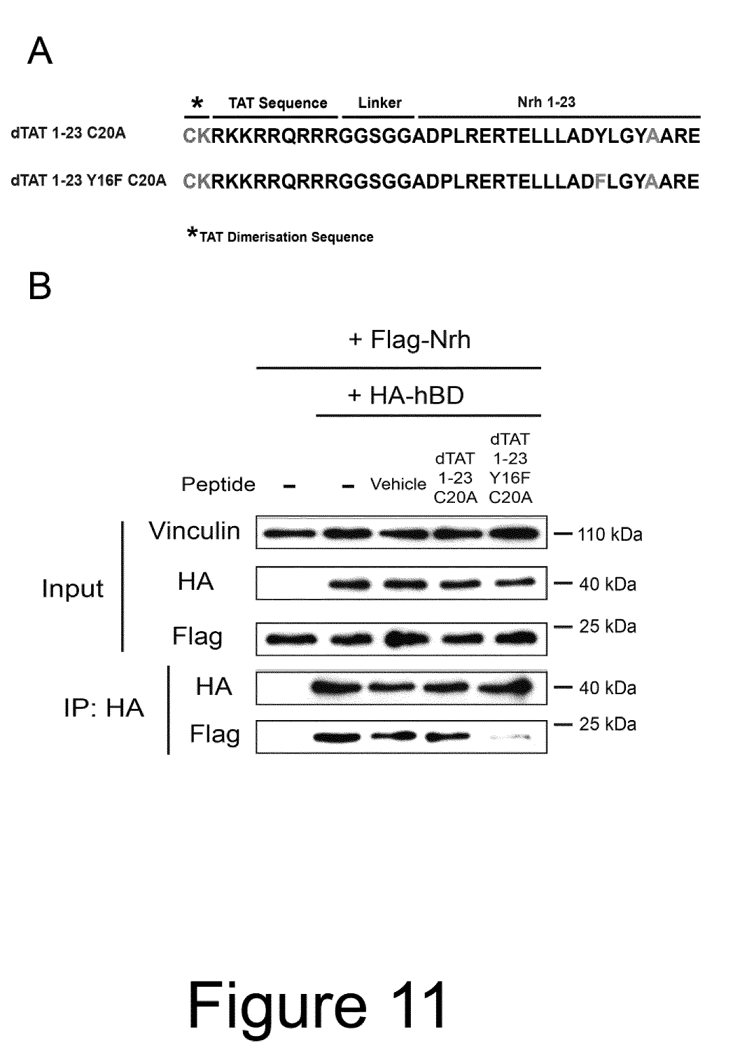

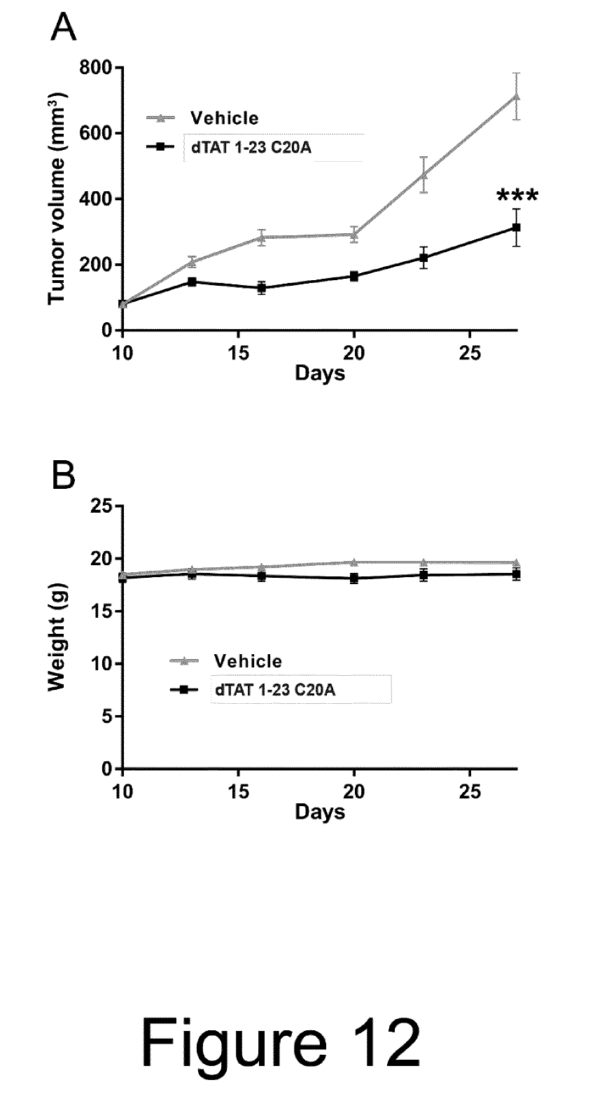

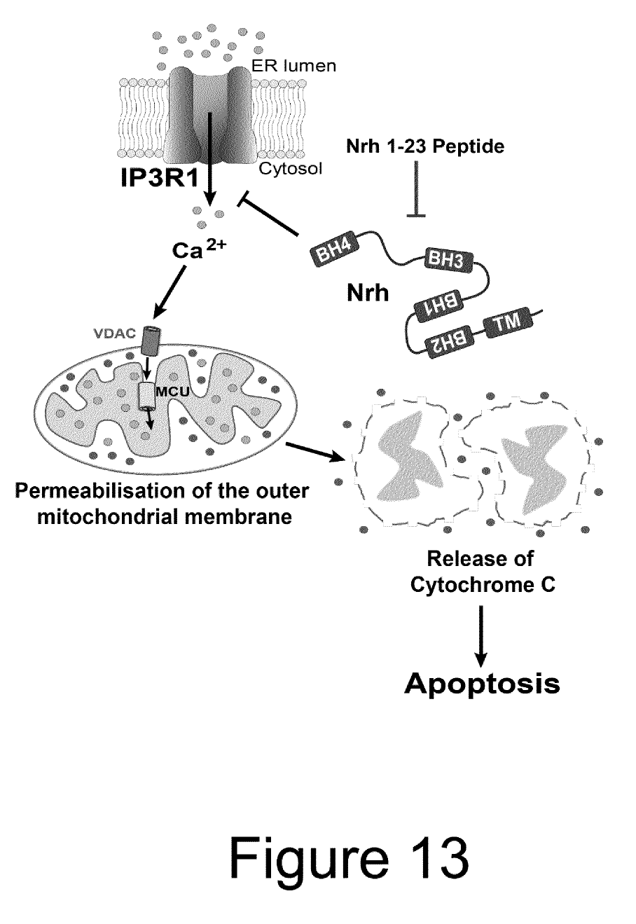

These various fusion proteins/mutated proteins are presented in FIG. 4A.

The effects of these various proteins were observed after transfection of HeLa cells by the empty vector pCS2+ or with the constructs coding for Nrh, Nrh-ActA, Nrh-cb5, dTM or dBH4.

The measurement of caspase activity in these cells, treated with 10 μM of thapsigargin for 24 h, is presented in FIG. 4B.

It appears that the caspase activity is greatly induced in the presence of thapsigargin, which corresponds to the expected effect. When the protein Bcl-2 L10/Nrh-WT is overexpressed in the cells, this effect is reduced by half; this effect is maintained in the presence of an Nrh protein targeted to the endoplasmic reticulum, or one which has had its membrane-anchoring domain deleted.

However, when the protein Bcl-2 L10/Nrh has a deletion of its BH4 domain, or when it is targeted to the mitochondrion, the apoptosis-blocking effect is no longer observed, caspase activity being maintained at the same percentage level as in the absence of this protein.

It may therefore be concluded that the localization of the protein Bcl-2 L10/Nrh at the endoplasmic reticulum, and the presence of its BH4 domain, are indispensable for obtaining the effect which blocks apoptosis induced by thapsigargin.

### Example 4. Construction of Various Peptides which May Potentially be Used as Competitive

inhibitors preventing the Bcl-2 L10/IP3R interaction, in the presence or absence of thapsigargin Several peptide constructs were produced from the peptide Nrh 1-23, the sequence of which is as follows: MADPLRERTELLLADYLGYCARE (SEQ ID NO. 2).

FIG. 5A presents the various point mutations carried out, in which the peptide Nrh 1-23 of sequence SEQ ID NO. 2 is mutated in order to create the peptides Nrh R6A (SEQ ID NO. 5), Nrh D15A (SEQ ID NO. 7), Nrh Y16F (SEQ ID NO. 6) and Nrh C20A (SEQ ID NO. 8), which are fused at the C-terminal end with the cytochrome b5 intracellular targeting domain (SEQ ID NO. 15).

FIG. 5B presents the ability of these different peptides to inhibit the interaction of Bcl-2 L10/Nrh with the BD domain of the receptor IP3R1. This interaction is quantified by the immunoprecipitation technique, the BD domain being tagged with an HA epitope and the protein Bcl-2 L10/Nrh being tagged with a Flag epitope.

HeLa cells are transfected with plasmids coding for Flag-Nrh, HA-hBD and the different point mutants of the peptide Nrh 1-23 Flag-tagged with a ratio of 1:2, and immunoprecipitation is carried out with an anti-HA antibody.

In the absence of peptide, the interaction between Flag-Nrh and HA-hBD is observed. However, when the plasmid coding for the peptide Nrh 1-23 (SEQ ID NO. 2), the peptide Nrh D15A (SEQ ID NO. 7) or Nrh C20A (SEQ ID NO. 8) are added, it is possible to see, on the Western blot in FIG. 5B, the absence of the band corresponding to the protein Flag-Nrh, demonstrating that these peptides dissociate the Nrh/IP3R1 interaction. On the other hand, the band corresponding to the protein Flag-Nrh is still visible, albeit faintly, in the presence of the peptides Nrh R6A (SEQ ID NO. 5) and Nrh Y16F (SEQ ID NO. 6).

The conclusion may therefore be drawn that the arginine residue at position 6 and the tyrosine residue at position 16 are critical for the biological function of the peptide Nrh 1-23 in dissociating the interaction between Nrh and IP3R1. The glutamine residue at position 15 and the cysteine residue at position 20 are not indispensable for the function of the peptide Nrh 1-23.

FIG. 5C shows the effects of the transfection of some of these various peptides into HeLa cells, the control cells being transfected with the empty vector pCS2+.

The measurement of caspase activity in these cells, treated with 10 μM of thapsigargin for 24 h, is presented in FIG. 5C.

It appears that in the control cells the caspase activity is induced in the presence of thapsigargin, which corresponds to the expected effect. When the peptide Nrh 1-23 (SEQ ID NO. 2) is present, and thus inhibits the Bcl-2 L10/IP3R1 interaction, this effect of the thapsigargin is potentiated, the caspase activity being virtually doubled; this effect is maintained in the presence of a peptide Nrh C20A, but disappears when the point mutation is carried out at the tyrosine Y16.

### Example 5. Dimerization of the Peptide Nrh and the Protein Bel-2 L10

FIG. 6 presents the experiments realized in order to demonstrate the ability of the peptide Nrh 1-23 to interact with the whole protein Nrh (Bcl-2 L10).

(A) HeLa cells were transfected with either empty vector pCS2+, Flag-Nrh, HA-hBD or HA-Beclin1 (referred to as HA-BECN1), a protein well known for its binding capacities to Nrh, then cellular extracts were incubated or not with 5 μg of biotin-labelled Nrh 1-23 wild-type peptide. Streptavidin is used for pulling-down the complexes comprising biotin-labelled Nrh peptide.

In FIG. 6A, it appears that Nrh 1-23 peptide is only able to interact with Nrh, but not with HA-hBD nor HA-BECN1.
(B) HeLa cells were transfected with either an empty vector pCS2+, Flag-Nrh, or Flag-Nrz (ortholog from zebrafish), then cellular extracts were incubated with (i) 5 μg of biotin-labelled Nrh 1-23 wild-type peptide or (ii) Nrh 1-23 R6A Y16F double mutant peptide. Streptavidin is used for pulling-down the complexes comprising biotin-labelled Nrh peptide.

It appears that Nrh 1-23 is only able to interact with Nrh, but not Nrz, thus exhibiting a level of species-specificity.

Moreover, the double mutant Nrh 1-23 R6A Y16F has a reduced interaction with Nrh. It can be concluded that at least one of these residues R6 and Y16 are critical for the Nrh/Nrh 1-23 interaction.

FIG. 7 presents the experiments realized in order to demonstrate the ability of the whole protein Nrh (Bcl-2 L10) to dimerise.

FIG. 7 illustrates the quantification of the interaction between Flag-Nrh and HA-Nrh using co-immunoprecipitation with anti-HA antibody. Formation of a Nrh homodimer is observed.

Nrh homodimerisation can be prevented by incubation with the Nrh 1-23 peptide, but not with the mutant peptide Nrh 1-23 Y16F, having lost its function through a point mutation in the critical Y16 residue.

### Example 6. Peptide Constructs of Competitive Inhibitor in Accordance with the Invention, or Mutated at the Tyrosine 16

FIG. 9A presents the peptide construct “TAT Nrh 1-23” as represented in sequence SEQ ID NO. 4; and a construct outside of the invention, in which the tyrosine 16 is not conserved, “TAT-Nrh 1-23 Y16F”, comprising the combination of the sequences of TAT (SEQ ID NO. 12), a spacer (SEQ ID NO. 14) and SEQ ID NO. 6 without the methionine.

FIG. 9B presents circular dichroism spectra of the peptides TAT-Nrh 1-23 and TAT-Nrh 1-23 Y16F, dissolved in a buffer [H₂O-0.1% trifluoroacetic acid] at a concentration of 80 μM in 20% trifluoroethanol.

The two peptide constructs have the characteristic signature of an alpha helix structure. Thus, it is shown that even though the point mutation Y16F does not affect the three-dimensional structure of the peptide, it does affect its biological function of competitive inhibitor.

### Example 7. Interaction Between the Endogenous Proteins Bcl-2 L10/Nrh and IP3R1 in the MDA-MB-231 Cells, in the Presence of the Peptide Constructs According to the Invention (TAT Nrh 1-23) or Outside of the Invention (TAT Nrh 1-23 Y16F)

FIG. 10A shows the results of Proximity Ligation Assay (PLA) experiments.

The MDA-MB-231 cells are incubated for 4 h with the peptide TAT Nrh-1-23 (SEQ ID NO. 4) or the control peptide TAT-Nrh 1-23 Y16F (SEQ ID NO 12, 14 and 6). After 3 washes of the cells, a 488 nm laser is used to excite the fluorophore (FITC) present in the endosomes, rupture the endosomal membrane and release the TAT-FITC peptides into the cytosol. The cells are incubated for 12 h then fixed, and the interaction between Nrh and IP3R1 is quantified by PLA using a rabbit anti-Nrh primary antibody and a mouse anti-IP3R1 primary antibody. This step is followed by incubation with a mixture of anti-rabbit and anti-mouse secondary antibodies, each coupled to a complementary nucleotide probe. A step of ligation then amplification is then carried out in the presence of fluorescent nucleotides, making it possible to amplify a specific signal in the case of spatial proximity of the probes. The fluorescence signal, in which each dot corresponds to an interaction in the cell, is recorded by means of a Nikon NiE fluorescence microscope. The images are quantified by the Image J software, and the interaction score is represented on the graph as number of dots per cell (N=3).

In FIG. 10A, showing the quantification of several PLA experiments, a score of 80±5 and 87±6 dots per cell is observed after incubation with the peptide dilution buffer (Vehicle) or the peptide TAT Nrh 1-23 in the absence of 488 nm flash, and hence in the absence of release of the peptides into the cytosol.

On the contrary, when a 488 nm flash is used to release the peptide TAT Nrh Y16F, the score falls to 65±5, then with the peptide TAT Nrh 1-23 to 11±1 dots per cell.

According to this quantification technique, the percentage inhibition of the Nrh/IP3R1 interaction is between 18.75% ((80-65)/80) and 25% ((87-65)/87) according to the negative control in question in the presence of the peptide outside of the invention. In the presence of the peptide TAT Nrh 1-23, the percentage inhibition of the Nrh/IP3R1 interaction is equal to 86% or 87%, as a function of the negative control in question.

It can therefore be concluded by this quantitative experiment that the TAT Nrh 1-23 peptide enables effective dissociation of the endogenous interaction between Nrh and IP3R1, corresponding to an inhibition of at least 30% of the Nrh/IP3R1 interaction, and that the TAT Nrh Y16F peptide only affects the interaction in a very limited way, by less than the limit value of 30%.

### Example 8. Biological Activity of the Peptide Constructs According to the Invention (TAT Nrh 1-23) or Outside the Invention (TAT Nrh 1-23 Y16F), in the Presence or Absence of Thapsigargin

The MDA-MB-231 cells, endogenously expressing the protein Bcl-2 L10, are incubated with the peptide TAT-Nrh 1-23 (SEQ ID NO. 4), or the peptide TAT-Nrh 1-23 Y16F (SEQ ID NO. 12, 14 and 6), for 36 h, following a flash of light or not, intended to rupture the endosomes containing the various peptides.

The results are presented in FIG. 10B.

When the peptides are released into the cytosol, the peptide construct according to the invention acts on the cells in combination with the thapsigargin and makes it possible to obtain a caspase activity of more than 60%.

### Example 9. Other Peptide Constructs According to the Invention (dTAT 1-23 C20A) or Outside of the Invention (dTAT 1-23 Y16F C20A)

Two synthetic peptides, represented in SEQ ID NO. 27 and 28, have been synthetized. These peptides present at the N-terminal end of the TAT internalization domain a Cystein (C) and a Lysine (K) residues, that allow the dimerization of the TAT sequence in oxidative conditions.

Since the dimeric TAT (dTAT) has enhanced endosomal escape properties, an efficient cell entry of the peptides is achieved without the use of an external physical agent, such as light, as used in example 7.

Both dTAT peptides have a TMR (tetramethylrhodamine) fluorescent group grafted to the side chain of the Lysine in position number 2 for tracking purpose.

The interaction between Flag-Nrh (Bcl-2 L10) and HA-hBD IP3R1 was assessed by co-immunoprecipitation in HeLa cells transfected with a plasmid coding for Flag-Nrh or HA-hBD IP₃R1, pre-incubated for 1 h at 37° C. in Ca²⁺-free Balanced Salt Solution (121 mM NaCl, 5.4 mM KCl, 0.8 mM MgCl₂, 6 mM NaHCO₃, 5.5 mM D-glucose, 25 mM HEPES, pH 7.3) with vehicle (peptide solvant [H2O-TFA 0.1%]), and:
10 μM of control peptide (dTAT 1-23 Y16F C20A) or
10 μM of wild-type peptide (dTAT 1-23 C20A).

Cells were washed twice and incubated for another 1 h at 37° C. before harvest.

As shown in FIG. 11B, in this experiment, dTAT 1-23 C20A but not dTAT 1-23 Y16F C20A disrupt the Nrh/IP3R1 complex, which means that they have been effectively introduced into the cells without any external agents.

This particular embodiment is advantageous over the previously used native TAT sequence in example 7, for peptide internalization.

### Example 10. In Vivo Effect of the Administration to Mice of the Peptide dTAT 1-23 C20A

SCID Mice have been used as tumor model for testing the effects of administration of the peptides according to the invention

MDA-MB-231 cancer cells have been injected into the mammary fat pad of SCID mice.

When the tumors reached a mean volume of about 90 mm³, mice were treated with administration of dTAT 1-23 C20A peptide (SEQ ID NO. 27, same peptide as shown in FIG. 11, but without the TMR fluorescent group) at 10 mg/kg, by peritumoral injection, once every three days, or administration of the vehicle alone (PBS) with the same injection periodicity.

Tumor volume was measured twice a week using calipers (mean±SEM; n=9 mice per condition; **, P<0.01), showing a significant effect of dTAT 1-23 C20A peptide in tumoral growth when used as an anti-tumoral monotherapy (FIG. 12A).

FIG. 12B shows the measurement of the weight of mice over the course of the experiments shown in FIG. 12A (mean±SEM; n=9 mice per condition). There is no difference in treated mice versus control mice, hence the treatment displays no apparent toxicity.

### BIBLIOGRAPHIC REFERENCES

### Patents

WO 2012/031103
WO 2013/128089
WO 2015/038662 Scientific Papers
1. Besbes S, Billard C. First MCL-1-selective BH3 mimetics as potential therapeutics for targeted treatment of cancer. Cell Death Dis. 2015 Jul. 9; 6
2. Arnaud E, Ferri K F, Thibaut J, Haftek-Terreau Z, Aouacheria A, Le Guellec D, Lorca T, Gillet G. The zebrafish bcl-2 homologue Nrz controls development during somitogenesis and gastrulation via apoptosis-dependent and -independent mechanisms. Cell Death Differ. 2006 July; 13(7): 1128-37.
3. Popgeorgiev N, Bonneau B, Ferri K F, Prudent J, Thibaut J, Gillet G. The apoptotic regulator Nrz controls cytoskeletal dynamics via the regulation of Ca2+ trafficking in the zebrafish blastula. Dev Cell. 2011 May 17; 20(5):663-76.
4. Bonneau B, Nougarède A, Prudent J, Popgeorgiev N, Peyriéras N, Rimokh R, Gillet G. The Bcl-2 homolog Nrz inhibits binding of IP3 to its receptor to control calcium signaling during zebrafish epiboly. Sci Signal. 2014 Feb. 11; 7(312)
5. Krajewska M, Kitada S, Winter J N, Variakojis D, Lichtenstein A, Zhai D, Cuddy M, Huang X, Luciano F, Baker C H, Kim H, Shin E, Kennedy S, Olson A H, Badzio A, Jassem J, Meinhold-Heerlein I, Duffy M J, Schimmer A D, Tsao M, Brown E, Sawyers A, Andreeff M, Mercola D, Krajewski S, Reed J C. Bcl-B expression in human epithelial and nonepithelial malignancies. Clin Cancer Res. 2008 May 15; 14(10):3011-21.
6. Cluzeau T, Robert G, Mounier N, Karsenti J M, Dufies M, Puissant A, Jacquel A, Renneville A, Preudhomme C, Cassuto J P, Raynaud S, Luciano F, Auberger P. BCL2L10 is a predictive factor for resistance to azacitidine in MDS and AML patients. Oncotarget. 2012 April; 3(4):490-501.
7. Rong Y P, Aromolaran A S, Bultynck G, Zhong F, Li X, McColl K, Matsuyama S, Herlitze S, Roderick H L, Bootman M D, Mignery G A, Parys J B, De Smedt H, Distelhorst C W. Targeting Bcl-2-IP3 receptor interaction to reverse Bcl-2's inhibition of apoptotic calcium signals. Mol Cell. 2008 Jul. 25; 31(2):255-65.
8. Monaco G, Beckers M, Ivanova H, Missiaen L, Parys J B, De Smedt H, Bultynck G. Profiling of the Bcl-2/Bcl-X(L)-binding sites on type 1 IP(3) receptor. Biochem Biophys Res Commun. 2012 Nov. 9; 428(1):31-5
9. Rong Y P, Bultynck G, Aromolaran A S, Zhong F, Parys J B, De Smedt H, Mignery G A, Roderick H L, Bootman M D, Distelhorst C W. The BH4 domain of Bcl-2 inhibits E R calcium release and apoptosis by binding the regulatory and coupling domain of the IP3 receptor. Proc Natl Acad Sci USA. 2009 Aug. 25; 106(34):14397-402.
10. Zhong F, Harr M W, Bultynck G, Monaco G, Parys J B, De Smedt H, Rong Y P, Molitoris J K, Lam M, Ryder C, Matsuyama S, Distelhorst C W. Induction of Ca²⁺-driven apoptosis in chronic lymphocytic leukemia cells by peptide-mediated disruption of Bcl-2-IP3 receptor interaction. Blood. 2011 Mar. 10; 117(10):2924-34.
12. Lavik A R, Zhong F, Chang M J, Greenberg E, Choudhary Y, Smith M R, McColl K S, Pink J, Reu F J, Matsuyama S, Distelhorst C W. A synthetic peptide targeting the BH4 domain of Bcl-2 induces apoptosis in multiple myeloma and follicular lymphoma cells alone or in combination with agents targeting the BH3-binding pocket of Bcl-2. Oncotarget. 2015 Sep. 29; 6(29):27388-402.
13. Monaco G, Decrock E, Akl H, Ponsaerts R, Vervliet T, Luyten T, De Maeyer M, Missiaen L, Distelhorst C W, De Smedt H, Parys J B, Leybaert L, Bultynck G. Selective regulation of IP3-receptor-mediated Ca2+ signaling and apoptosis by the BH4 domain of Bcl-2 versus Bcl-Xl. Cell Death Differ. 2012 February; 19(2):295-309
14. Mikoshiba K. IP3 receptor/Ca2+ channel: from discovery to new signaling concepts. J Neurochem. 2007 September; 102(5):1426-46. Review.
15. Yoshikawa F, Morita M, Monkawa T, Michikawa T, Furuichi T, and Mikoshiba K (1996) Mutational analysis of the ligand binding site of the inositol 1,4,5-trisphosphate receptor. J Biol Chem. 271, 18277-18284.
16. Xiaoying Chen, Jennica Zaro, and Wei-Chiang Shen. Fusion Protein Linkers: Property, Design and Functionality. Adv Drug Deliv Rev. 2013 Oct. 15; 65(10): 1357-1369.

1.-14. (canceled)

15. A process for treating a cancer, the cells of which express the protein Bcl-2 L10, comprising a step of administering a competitive inhibitor of the binding of the protein Bcl-2 L10 to the ligand binding domain of at least one of the IP3R receptors to a patient suffering from said cancer.

16. The process according to claim 15, wherein said competitive inhibitor comprises a peptide domain, the sequence of which has at least 80% identity with the sequence SEQ ID NO:1 [RERTELLLADY], the underlined arginine and tyrosine residues being conserved.

17. The process according to claim 16, wherein said competitive inhibitor comprises a peptide domain, the sequence of which has at least 80% identity with one of the sequences SEQ ID NO:2 [MADPLRERTELLLADYLGYCARE] or SEQ ID NO:3 [ADPLRERTELLLADYLGYCARE], the underlined arginine and tyrosine residues being conserved.

18. The process according to claim 16, wherein said competitive inhibitor is constructed according to the following structure: [ADR]ₓ-[ESP]_{y}-[DOM]-[SADR]_{z}, in which:
ADR is a targeting compound,
ESP is a spacer,
DOM is the peptide domain defined in claim 16,
SADR denotes a specific intracellular targeting peptide domain,
wherein x, y, and z are equal to 0 or 1 independently of one another, and the sum (x+y+z) is equal or superior to 1.

19. The process according to claim 18, wherein in said competitive inhibitor, the domains ADR, ESP, and SADR are peptide domains.

20. The process according to claim 15, wherein said competitive inhibitor consists of a peptide having the peptide sequence represented by an amino acid sequence selected among the group consisting of: SEQ ID NO:4, SEQ ID NO:18, and SEQ ID NO:27.

21. The process according to claim 15, comprising furthermore a step of administration of at least a second active agent and/or a step of the implementation of any conventional method for treating cancer, to the patient suffering from said cancer.

22. The process according to claim 21, wherein said second active agent is a chemotherapy product or immunotherapy product.

23. The process according to claim 21, wherein said conventional method for treating cancer is surgery or radiotherapy.

24. The process according to claim 15, wherein said competitive inhibitor further inhibits the homodimerization of Bcl-2 L10.

25. A competitive inhibitor of the binding of the protein Bcl-2 L10 to the ligand binding domain of at least one of the IP3R receptors, constructed according to the following structure: [ADR]ₓ-[ESP]_{y}-[DOM]-[SADR]_{z}, in which
ADR is a targeting compound,
ESP is a spacer,
DOM is the peptide domain defined in claim 16,
SADR denotes a specific intracellular targeting peptide domain,
wherein x, y, and z are equal to 0 or 1 independently of one another, and the sum (x+y+z) is equal or superior to 1.

26. An inhibitor according to claim 25, characterized in that the domains ADR, ESP, and SADR are peptide domains.

27. An inhibitor according to claim 26, comprising an amino acid sequence represented by an amino acid sequence selected among the group consisting of: SEQ ID NO:4, SEQ ID NO:18, and SEQ ID NO:27.

28. A pharmaceutical composition comprising, in a pharmaceutically acceptable medium, at least one inhibitor according to claim 25.

29. The process according to claim 17, wherein said competitive inhibitor is constructed according to the following structure: [ADR]ₓ-[ESP]_{y}-[DOM]-[SADR]_{z}, in which:
ADR is a targeting compound,
ESP is a spacer,
DOM is the peptide domain defined in claim 16,
SADR denotes a specific intracellular targeting peptide domain,
wherein x, y, and z are equal to 0 or 1 independently of one another, and the sum (x+y+z) is equal or superior to 1.

30. The process according to claim 29, wherein in said competitive inhibitor, the domains ADR, ESP, and SADR are peptide domains.

31. The process according to claim 16, wherein said competitive inhibitor is constructed according to the following structure: [ADR]ₓ-[ESP]_{y}-[DOM]-[SADR]_{z}, in which:
ADR is a targeting compound,
ESP is a spacer,
DOM is the peptide domain defined in claim 17,
SADR denotes a specific intracellular targeting peptide domain,
wherein x, y, and z are equal to 0 or 1 independently of one another, and the sum (x+y+z) is equal or superior to 1.

32. A competitive inhibitor of the binding of the protein Bcl-2 L10 to the ligand binding domain of at least one of the IP3R receptors, constructed according to the following structure: [ADR]ₓ-[ESP]_{y}-[DOM]-[SADR]_{z}, in which
ADR is a targeting compound,
ESP is a spacer,
DOM is the peptide domain defined in claim 17,
SADR denotes a specific intracellular targeting peptide domain,
wherein x, y, and z are equal to 0 or 1 independently of one another, and the sum (x+y+z) is equal or superior to 1.

33. The process according to claim 17, wherein said competitive inhibitor is constructed according to the following structure: [ADR]ₓ-[ESP]_{y}-[DOM]-[SADR]_{z}, in which:
ADR is a targeting compound,
ESP is a spacer,
DOM is the peptide domain defined in claim 17,
SADR denotes a specific intracellular targeting peptide domain,
wherein x, y, and z are equal to 0 or 1 independently of one another, and the sum (x+y+z) is equal or superior to 1.

34. The process according to claim 31, wherein in said competitive inhibitor, the domains ADR, ESP, and SADR are peptide domains.

35. An inhibitor according to claim 32, characterized in that the domains ADR, ESP, and SADR are peptide domains.

36. An inhibitor according to claim 35, comprising an amino acid sequence represented by an amino acid sequence selected among the group consisting of: SEQ ID NO:4, SEQ ID NO:18, and SEQ ID NO:27.

37. A pharmaceutical composition comprising, in a pharmaceutically acceptable medium, at least one inhibitor according to claim 32.

38. The process according to claim 33, wherein in said competitive inhibitor, the domains ADR, ESP, and SADR are peptide domains.
